(19) United States
(12) Patent Application Publication
(10) Pub. No.: US 2017/0056179 A1
(43) Pub. Date: Mar. 2, 2017

(54) EXPANDABLE INTERVERTEBRAL CAGE WITH LIVING HINGES APPARATUS, SYSTEMS AND METHODS OF MANUFACTURE THEREOF

(71) Applicant: Morgan Packard Lorio, Bristol, TN (US)

(72) Inventor: Morgan Packard Lorio, Bristol, TN (US)

(21) Appl. No.: 14/839,991

(22) Filed: Aug. 30, 2015

(51) Int. Cl. A61F 2/30; B23K 15/00; B22F 3/105; A61F 2/44; B29C 67/00

(52) U.S. Cl. CPC A61F 2/3094; A61F 2/30942; A61F 2/4455; B29C 67/0051; B22F 3/1055; B23K 15/0086; B23K 15/0093; B33Y 10/00

(57) ABSTRACT

An expandable intervertebral cage with living hinges manufactured using 3D printing. The intervertebral cage is configured to expand from an unexpanded to an expanded configuration. The intervertebral cage can include a deployment system, such as a variable volume pouch or deployment cable, to apply force to the intervertebral cage to deploy the intervertebral cage.

### CROSS REFERENCE TO RELATED APPLICATIONS

The present application is related to: U.S. patent application Ser. No. 13/248,747 filed Sep. 29, 2011, entitled INTERVERTEBRAL DEVICE AND METHODS OF USE, which claims priority to U.S. Provisional Application No. 61/389,986; U.S. patent application Ser. No. 14/422,750 filed Feb. 20, 2015, entitled INTERVERTEBRAL CAGE APPARATUS AND SYSTEM AND METHODS OF USING THE SAME, which is a national stage application under 35 U.S.C. §371 of PCT Application No. PCT/US2013/056500, filed Aug. 23, 2013, which claims priority to U.S. Provisional Application No. 61/693,738 filed Aug. 27, 2012 and U.S. Provisional Application No. 61/778,271 filed Mar. 12, 2013; and PCT Application No. PCT/US2014/018772, filed Feb. 26, 2014, entitled VERTICALLY EXPANDABLE INTERVERTEBRAL CAGE, DEPLOYMENT DEVICES, AND METHODS OF USING THE SAME, which claims priority to U.S. Provisional Application No. 61/778,220 filed Mar. 12, 2013, the entire contents of all applications are hereby expressly incorporated by reference.

### FIELD

The present invention relates generally to an expandable intervertebral cage or implant used in spinal fusion procedures and, more specifically relates to apparatus, systems and methods for manufacturing the expandable intervertebral cage using 3D printing methods.

### BACKGROUND

Vertebrae are the individual irregular bones that make up the spinal column. There are normally thirty-three vertebrae in humans, including the five that are fused to form the sacrum (the others are separated by intervertebral discs) and the four coccygeal bones which form the tailbone. The upper three regions comprise the remaining 24, and are grouped under the names cervical (7 vertebrae), thoracic (12 vertebrae) and lumbar (5 vertebrae), according to the regions they occupy. This number is sometimes increased by an additional vertebra in one region, or it may be diminished in one region, the deficiency often being supplied by an additional vertebra in another. The number of cervical vertebrae is, however, very rarely increased or diminished.

A typical vertebra consists of two essential parts: an anterior (front) segment, which is the vertebral body; and a posterior part—the vertebral (neural) arch which encloses the vertebral foramen. The vertebral arch is formed by a pair of pedicles and a pair of laminae, and supports seven processes, four articular, two transverse, and one spinous, the latter also being known as the neural spine.

When the vertebrae are articulated with each other, the bodies form a strong pillar for the support of the head and trunk, and the vertebral foramina constitute a canal for the protection of the medulla spinalis (spinal cord), while between every pair of vertebrae are two apertures, the intervertebral foramina, one on either side, for the transmission of the spinal nerves and vessels.

Conventional spinal cage assemblies are used in spinal fusion procedures to stabilize vertebrae. Spinal fusion typically employs the use of cage assemblies that space apart and fuse together adjacent vertebrae. Spinal fusion techniques include removing disc material which separates the vertebrae and impacting bone into the disc area. These cage assemblies are generally hollow and include openings in the side thereof to provide access for bone to grow. These cages are often formed of titanium and are available in varying shapes and sizes.

The current intervertebral or interbody cages are designed using 3 major principles; the anatomical limitations of the surgical approach, optimization of bone graft volume to promote bony fusion, and optimization of the device contact with the vertebral endplates to resist subsidence. The current cages are generally static in that they cannot change shape or volume, thus they are limited by the anatomy and technique, and therefore they do not provide optimal bone graft volume or surface contact.

Many conventional spinal cage assemblies use parallel distraction of opposing vertebrae prior to placing an implant. However, not all vertebrae are in parallel opposition. A normal and healthy spine has a natural curvature referred to as lordosis. As a result of the curvature, opposing vertebrae are positioned with their end plates in non-parallel alignment depending upon the position in the spine.

A need exists for an intervertebral cage or implant that can expand to change shape and/or volume provide optimal bone graft volume or surface contact to maintain or achieve a desired lordosis between opposing vertebrae. The present invention attempts to solve these problems as well as others.

### SUMMARY

Certain embodiments of the present application relate to expandable intervertebral cages and methods of making and using the sameSome embodiments relate to an intervertebral cage that can be configured for positioning between two vertebrae and specifically between two vertebral end plates including but not limited to patient specific intervertebral cages.

Disclosed herein is a method of manufacturing the expandable intervertebral cages by additive manufacturing, also known as 3D printing. The expandable intervertebral cage can be molded in a single integral piece and constructed layer-by-layer, bottom-to-top, such that the components are integrally connected by a living hinge.

In one aspect of the present invention, a method for making an expandable intervertebral cage with living hinges using 3D printable materials for placement between adjacent vertebrae, the method including providing 3D data of the expandable intervertebral cage to a 3D printer, the expandable intervertebral cage includes a circuitous body having a plurality of side segments rotatably attached by integral living hinges configured to flex or deform during the transition of the circuitous body from an unexpanded configuration to an expanded configuration, and printing the plurality of side segments and integral living hinges of the circuitous body using one or more 3D printable materials.

In some embodiments, the one or more 3D materials is selected from the group consisting of: thermoplastics, photopolymers, metal powders, eutectic metals, titanium alloys and combinations thereof. In some embodiments, the one or more 3D material is selected from the group consisting of: a natural biocompatible material, a synthetic biocompatible material, a metallic biocompatible material, adaptive material, 4D printing, and combinations thereof. In some embodiments, the one or more 3D material is selected from the group consisting of: polyetherketone (PEK), polyetherimide (PEI), such as Ultem, ultrahigh molecular weight polyethylene (UHMPE), polyphenylene, polyether-ether-ketone (PEEK), comprise a memory PEEK material such as, for example, PEEK Altera, and combinations thereof. In some embodiments, the relatively flexible 3D printable material comprises a non-metallic material and the relatively rigid 3D printable material comprises a metallic material.

In some embodiments, the intervertebral cage includes an external coating selected from the group consisting of: plasma spray, hydroxyapatite coating, biologics, antibiotics, drug or gene therapy, nanotechnology platform(s), and combinations thereof.

In some embodiments, the integral living hinges comprise a relatively flexible 3D printable material and the side segments comprise a relatively rigid 3D printable material.

In some embodiments, the 3D data is predefined data of standard cage sizes. In some embodiments, the 3D data is 3D imaging data, the method further comprising pre-operatively imaging adjacent vertebrae of a patient to generate 3D imaging data.

In some embodiments, the living hinges comprise narrowed portions of the circuitous body and/or cutouts into the circuitous body configured to allow localized flexure or deformations of the circuitous body.

In some embodiments, the circuitous body includes proximal and distal ends oppositely disposed along a lateral axis and in the unexpanded configuration the proximal and distal ends are at a maximum separation and in the expanded configuration the proximal and distal ends are closer together, the expandable intervertebral cage configured to horizontally expand from the unexpanded configuration to the expanded configuration between adjacent vertebrae.

In some embodiments, the circuitous body includes a top panel and a bottom panel, each top and bottom panel rotatably attached by integral living hinges to one or more side segments, the expandable intervertebral cage configured to vertically expand from the unexpanded configuration to the expanded configuration between adjacent vertebrae.

In some embodiments, the expandable intervertebral cage further comprises a variable volume pouch positionable within an interior volume of the intervertebral cage.

In some embodiments, the expandable intervertebral cage further comprises a deployment cable coupled to the circuitous body and configured to apply a force to the circuitous body to transition the circuitous body from the unexpanded configuration to the expanded configuration.

In some embodiments, the expandable intervertebral cage further comprises a deployment tool coupled to the circuitous body and configured to apply a force to the circuitous body to transition the circuitous body from the unexpanded configuration to the expanded configuration.

In some embodiments, the expandable intervertebral cage is configured for positioning between end plates of two vertebrae and further configured to transition from an unexpanded configuration to an expanded configuration resulting in a change of the dimensions and shape of the expandable intervertebral cage and increasing a modifiable interior volume of the expandable intervertebral cage.

In another aspect of the present invention, a method for making and using a patient specific expandable intervertebral cage with living hinges using 3D printable materials for placement between adjacent vertebrae, the method including pre-operatively imaging adjacent vertebrae of the patient to generate 3D imaging data, providing the 3D data to a 3D printer, printing an expandable intervertebral cage using one or more 3D printable materials, the expandable intervertebral cage includes a circuitous body having a plurality of side segments rotatably attached by integral living hinges configured to flex or deform during the transition of the circuitous body from an unexpanded configuration to an expanded configuration, surgically positioning the expandable intervertebral cage between the adjacent vertebrae, and expanding the expandable intervertebral cage from the unexpanded configuration to the expanded configuration between the adjacent vertebrae.

In some embodiments, the one or more 3D material is selected from the group consisting of: thermoplastics, photopolymers, metal powders, eutectic metals, titanium alloys, a natural biocompatible material, a synthetic biocompatible material, a metallic biocompatible material, adaptive materials, 4D printing, polyetherketone (PEK), polyetherimide (PEI), such as Ultem, ultrahigh molecular weight polyethylene (UHMPE), polyphenylene, polyether-ether-ketone (PEEK), comprise a memory PEEK material such as for example, PEEK Altera, and combinations thereof.

In a further aspect of the present invention, a system for deploying an expandable intervertebral cage with living hinges using 3D printable materials for placement between adjacent vertebrae, the system including an expandable intervertebral cage made of one or more 3D printable materials configured to transition from an unexpanded configuration to an expanded configuration, having a proximal end and a distal end, the expandable intervertebral cage includes a circuitous body having a plurality of side segments rotatably attached by integral living hinges configured to flex or deform during the transition of the circuitous body from the unexpanded configuration to the expanded configuration, and a variable volume pouch positionable within the intervertebral cage, the variable volume pouch being configured to move the expandable intervertebral cage from the unexpanded configuration to the expanded configuration.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through the use of the accompanying drawings.

FIG. 1A is a perspective view of one embodiment of an intervertebral cage in an unexpanded position.

FIG. 1B is a top view of one embodiment of an intervertebral cage in an unexpanded configuration.

FIG. 2A is a perspective view of one embodiment of an intervertebral cage in an expanded configuration.

FIG. 2B is a top view of one embodiment of an intervertebral cage in an expanded configuration.

FIG. 3 is a flowchart of a 3D model printing method.

FIG. 4A is a perspective view of one embodiment of a variable volume pouch in an unexpanded configuration.

FIG. 4B is a perspective view of one embodiment of a variable volume pouch in an expanded configuration.

FIG. 5A is a perspective view of one embodiment of an intervertebral cage apparatus including an intervertebral cage and a variable volume pouch in an unexpanded configuration.

FIG. 5B is a side view of one embodiment of a plug.

FIG. 6A is a perspective view of one embodiment of an intervertebral cage apparatus including an intervertebral cage and a variable volume pouch in an expanded configuration.

FIG. 6B is a top cutaway view of one embodiment of an intervertebral cage apparatus in an expanded configuration.

FIG. 7 is a perspective view of one embodiment of an intervertebral cage apparatus including a variable volume pouch and an intervertebral cage having a lateral split in an expanded configuration.

FIG. 8 is a schematic illustration of one embodiment of a deployment system.

FIG. 9 is a perspective view of one embodiment of an intervertebral cage apparatus including an intervertebral cage and a deployment cable in an unexpanded configuration.

FIG. 10A is a perspective view of one embodiment of an intervertebral cage apparatus including an intervertebral cage and a deployment cable in an expanded configuration.

FIG. 10B is a top view of one embodiment of an intervertebral cage apparatus including an intervertebral cage and a deployment cable in an expanded configuration.

FIG. 11 is a perspective view of one embodiment of an intervertebral cage apparatus including a variable volume pouch, a deployment cable, and an intervertebral cage having a lateral split in an expanded configuration.

FIG. 12 is a flowchart illustrating one embodiment of process for using the intervertebral cage and/or the intervertebral cage apparatus.

FIG. 13 is a flowchart illustrating one embodiment of a method of using a variable volume pouch and an intervertebral cage as part of an intervertebral cage apparatus.

FIG. 14 is a flowchart illustrating one embodiment of a method of using an intervertebral cage apparatus including an intervertebral cage and a deployment cable.

FIG. 15A is a perspective view of one embodiment of an intervertebral cage apparatus having a distal aperture in an unexpanded position.

FIG. 15B is a cross-sectional view of one embodiment of an intervertebral cage apparatus along section A-A as shown in FIG. 15A.

FIG. 15C is a cross-sectional view of one embodiment of an intervertebral cage apparatus along section B-B as shown in FIG. 15B showing a slot on a distal aperture.

FIG. 16A is a partial cross-sectional view of one embodiment of an implantation tool which can be used to convert an intervertebral cage apparatus from an unexpanded position to an expanded position.

FIG. 16B is an enlarged view of a distal end of one embodiment of an implantation tool focused on Section A-A as shown in FIG. 16A.

FIG. 16C is a view from the distal end of one embodiment of an implantation tool showing the distal end of an intervertebral cage apparatus having a connector, teeth, and shaft.

FIG. 17 is a cross-sectional view of one embodiment of the intervertebral cage apparatus having a distal aperture and one embodiment of an implantation tool prior to being connected.

FIG. 18 is a perspective view of one embodiment of a vertically expandable intervertebral cage.

FIG. 19A is a side elevation view of the intervertebral cage of FIG. 18 in a first position.

FIG. 19B is a side elevation view of the intervertebral cage of FIG. 18 in a second position.

FIG. 19C is a side elevation view of the intervertebral cage of FIG. 18 in a third position.

### DETAILED DESCRIPTION

The words proximal and distal are applied to denote specific ends of components of the current invention described herein. A proximal end refers to the end of a component nearer to a medical professional when the component is implanted. A distal end refers to the end of a component further from the medical professional when the component is implanted.

Although some details of an expandable intervertebral cage are provided herein, further details can be found in U.S. Publication No. 2012/0083887 published on Apr. 5, 2012, entitled “Intervertebral Device and Methods of Use,” in U.S. Publication No. 2012/0083889 published on Apr. 5, 2012, entitled “Intervertebral Device and Methods of Use,” and in International Publication No. WO2014/158619 published on Oct. 2, 2014, entitled “Vertically Expandable Intervertebral Cage, Deployment Devices, And Methods Of Using The Same”, all of which are incorporated herein in their entirety by reference.

The present application discloses embodiments of an expandable intervertebral cage that can be manufactured by additive manufacturing, also known as three-dimensional printing or 3D printing. 3D printing is a high-speed additive manufacturing technology that can deposit various types of materials in powder, liquid, or granular form in a printer-like fashion. Deposited layers can be cured layer by layer or, alternatively, for granular deposition, an intervening adhesive step can be used to secure layered granules together in bed of granules and the multiple layers subsequently can be cured together for example, with laser or light curing.

As used herein, 3D printing includes many types of printing technologies, such as additive manufacturing, rapid manufacturing, layered manufacturing, rapid prototyping, laser sintering, electron beam melting (EBM), etc. 3D printing also includes adaptive materials and 4D printing. In some embodiments, 4D printing adds time to the length, width, and height of the intervertebral cage printed using additive manufacturing. With the addition of time, the intervertebral cages can become adaptable, changing structures: self-evolving structures.

One feature using a 3D printing process is the recognition that the expandable intervertebral cage does not have to be limited to the traditional manufacturing constraints such as those imposed by conventional machining or casting methods. The 3D printing process of the expandable intervertebral cage can use various materials including thermoplastics, photopolymers, metal powders, eutectic metals, titanium alloys and other materials.

The 3D printing process of the expandable intervertebral cage can use various materials including thermoplastics, photopolymers, metal powders, eutectic metals, titanium alloys and other materials. Because the expandable intervertebral cage will be implanted in the body, the preferred material is a biocompatible material, for example, a natural biocompatible material, a synthetic biocompatible material, a metallic biocompatible material, adaptive material, and/or any other desired biocompatible material. The implant can be made of polyetherketone (PEK), polyetherimide (PEI), such as Ultem, ultrahigh molecular weight polyethylene (UHMPE), polyphenylene, polyether-ether-ketone (PEEK), comprise a memory PEEK material such as, for example, PEEK Altera, or any other desired biocompatible material. The 3D printing process may also include hybrid material designs, i.e. PEEK & Titanium.

In some embodiments the expandable intervertebral cage may be manufactured using a 3D printing process with a metal material having a relatively high density deposited within a polymer substrate having a relatively low density. In some embodiments the expandable intervertebral cage may be manufactured with the side segments made from a relatively rigid 3D printable material and the integral living hinges made from a relatively flexible 3D printable material. Other combinations may include any combination of metal, polymer, ceramic, or composites for the side segments and integral living hinge. In some embodiments the expandable intervertebral cage may be manufactured using a combination of traditional manufacturing processes and 3D printing processes.

The intervertebral cage may also include an external coating, for example, plasma spray, hydroxyapatite coating, biologics, antibiotics, drug or gene therapy, nanotechnology platform(s), etc. The coating may be part of the 3D printing process or secondarily applied or integrated into the implant.

The 3D printing process of the expandable intervertebral cage may also be directed toward patient-specific and/or patient-engineered implants. With such systems, the expandable intervertebral cage may be designed from non-invasive imaging data taken of the patient undergoing the surgical procedure pre-operatively and manufactured using this anatomical information to accommodate the individual anatomy of the patient. By doing this, stock cages do not have to be stockpiled in multiple sizes prior to surgery, as is currently done, saving time and money.

The intervertebral cages described herein can be “space-agnostic” in the sense that it can be used for multiple spinal fusion techniques including anterior lumbar interbody fusion (ALIF), posterior lumbar interbody fusion (PLIF), transforaminal lumbar interbody fusion (TLIF), lateral lumbar interbody fusion (XLIF), or applied from any direction, including transosseous.

The present invention relates generally to interbody spinal fusion implants, and in particular to intervertebral cages configured to restore and maintain two adjacent vertebrae of the spine in correct anatomical angular relationship. The intervertebral cages are configured for positioning between end plates of two vertebrae in an unexpanded configuration and then expand to an expanded configuration that results in a change of the dimensions and shape of the intervertebral cage. The intervertebral cages the help in fusing the opposing vertebrae while maintaining lordosis.

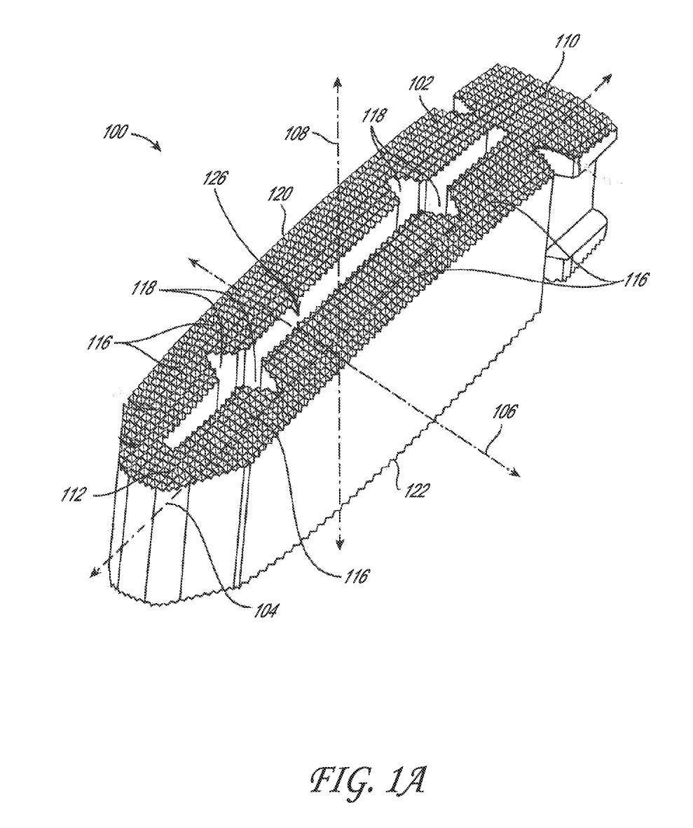

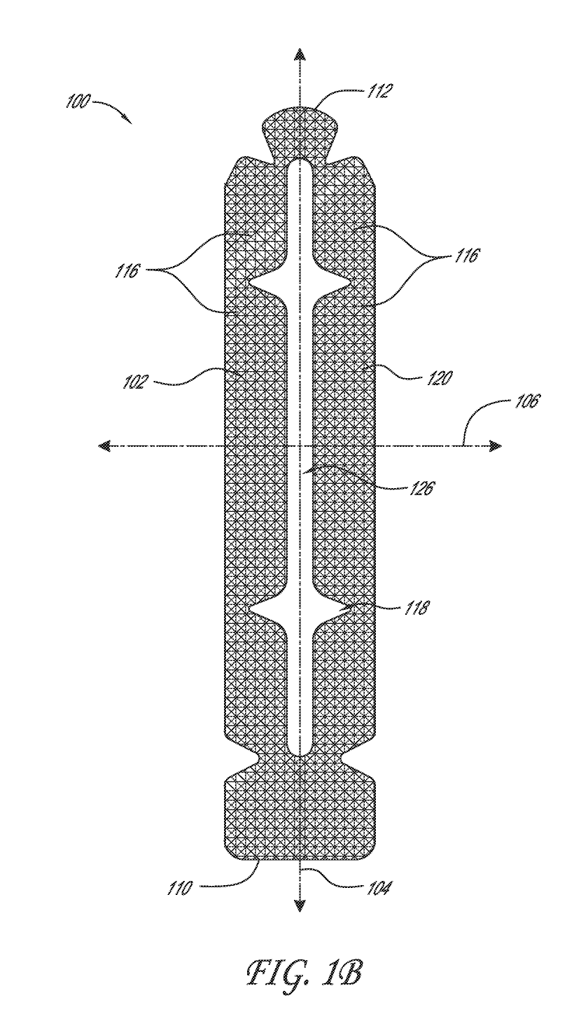

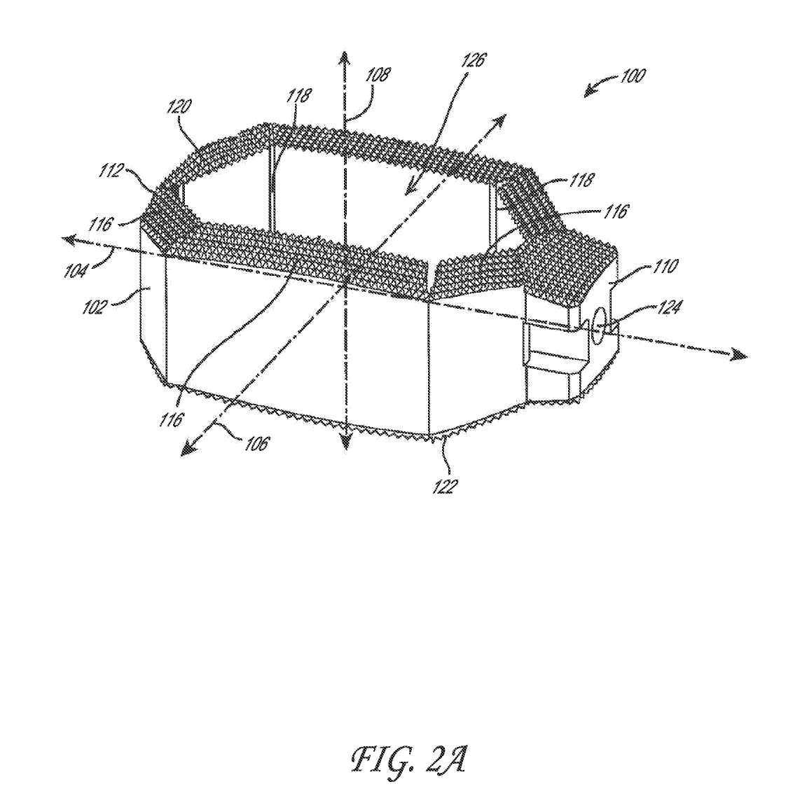

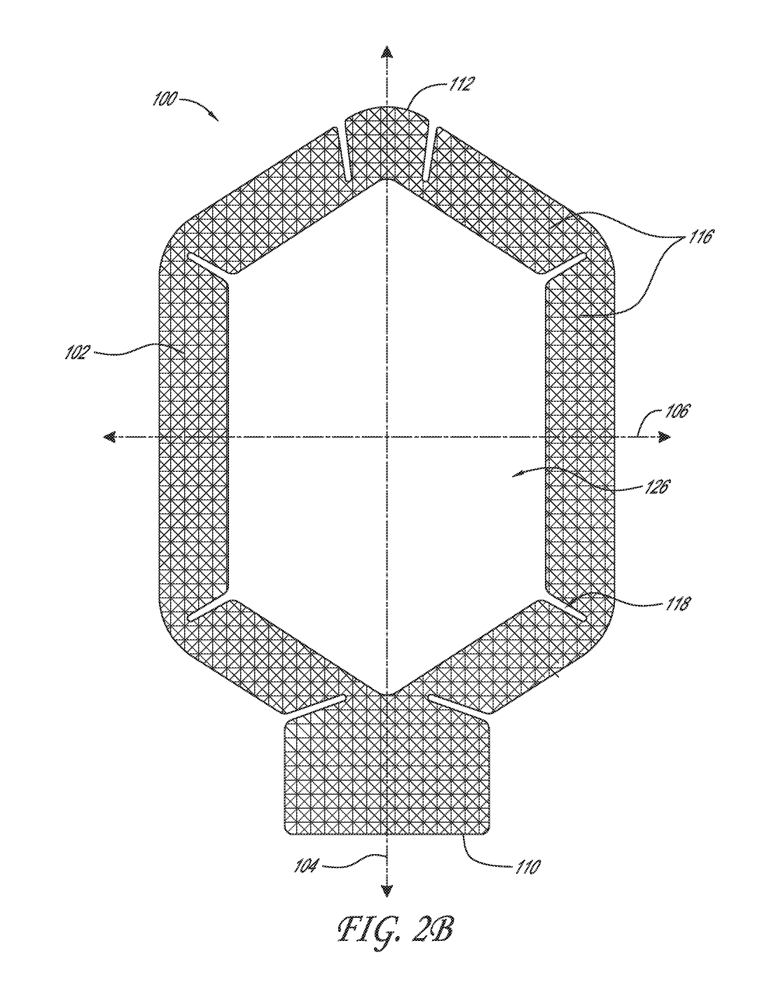

FIGS. 1A and 1B and in FIGS. 2A and 2B show one embodiment of an expandable intervertebral cage 100. The intervertebral cage 100 can be configured for positioning between two vertebrae and specifically for positioning between the end plates of two vertebrae for fusing the opposing vertebrae. The intervertebral cage 100 can be positioned in an unexpanded configuration as depicted in FIGS. 1A and 1B and can be positioned in an expanded configuration as depicted in FIGS. 2A and 2B. In some embodiments, and as depicted in FIGS. 1A and 1B and 2A and 2B, the change of the intervertebral cage 100 from an unexpanded configuration to an expanded configuration can result in a change of the dimensions and shape of the intervertebral cage 100.

The intervertebral cage 100 can comprise a circuitous body 102 defining a perimeter and an internal volume. The body 102 can be configured to contact the two vertebrae between which the intervertebral cage 100 is positioned and/or to transfer force from one of the vertebrae between which the intervertebral cage 100 is positioned to the other of the vertebrae between which the intervertebral cage is positioned. The body 102 can comprise a variety of shapes and sizes that approximate the dimensions between the two vertebrae and can be made from a variety of materials.

As seen in FIGS. 1A and 1B and in FIGS. 2A and 2B, embodiments of the body 102 can comprise segments 116 rotatably connected to each other by integral flexible connectors or living hinges 118 to form a modifiable inner volume 126. The living hinges 118 need not be true hinges, in that they need not be capable of being flexed many times, but instead may only be robust enough to bend once from the unexpanded configuration to a expanded configuration.

The living hinges 118 can, in some embodiments, be located on an interior surface of the body 102 proximate to the internal volume 126, and in some embodiments, the living hinges 118 can be located on an exterior surface of the body 102. In some embodiments, the living hinges 118 can comprise portions of the body 102 that are configured to bend. In some embodiments, the living hinges 118 can be discrete elements in that the bending may be localized in one or several positions on the body 102, and in some embodiments, the living hinges 118 may be non-discrete elements in that the bending may not be localized, but rather occur over all or large portions of the body 102. In some embodiments in which the living hinges 118 comprise discrete elements, the flexible connector can comprise a shape, a feature, a material characteristic, or any other aspect that concentrates stresses and/or deformation. As specifically depicted in FIGS. 1A and 1B and 2A and 2B, in some embodiments, the living hinges 118 can comprise narrowed portions of the body 102 and/or cutouts into the body 102 to allow localized deformations of the body 102 when the body 102 is moved from an unexpanded configuration to an expanded configuration.

The body 102 can have a proximal end 110 and a distal end 112. In some embodiments, the proximal end 110 and/or the distal end 112 can be an integral part of the body 102 and can partially define the internal volume of the body 102. In some embodiments, and as seen in FIG. 2A, the proximal end 110 of the body 102 can comprise a proximal aperture 124. In some embodiments, for example, the proximal aperture 124 can extend through the proximal end 110 of the body 102 and into the internal volume 126 of the body 102. Advantageously, in embodiments in which the proximal aperture 124 extends through the proximal end 110 of the body 102 and into the internal volume 126 of the body 102, the proximal aperture 124 can provide access to the internal volume 126 and/or components or features of an intervertebral cage apparatus located within the internal volume 126. The proximal end 110 can comprise a variety of shapes and sizes. Similarly, the proximal aperture 124 can comprise a variety of shapes and sizes.

The distal end 112 of the body 102 can be configured to facilitate insertion of the intervertebral cage 100 between the vertebrae. In some embodiments, for example, the distal end 112 of the body 102 can comprise a tapered and/or pointed shape to facilitate insertion of the body 102 into the space between the vertebrae. Advantageously, such a tapered and/or pointed shape to the distal end of the body 102 can facilitate in achieving adequate separation between the vertebrae and/or can minimize the insertion force required to insert the body 102 of the intervertebral cage 100 into the space between the vertebrae.

As seen in FIG. 1A, a longitudinal axis 104 of the body 102 can extend between the proximal end 110 and the distal end 112 of the body. As further seen in FIG. 1A, the body 102 can comprise a top 120 and a bottom 122. In some embodiments, the top 120 and the bottom 122 can each be configured for interaction with one of the vertebrae between which the intervertebral cage 100 is positioned, and specifically for interaction with one of the end plates of one of the vertebrae between which the intervertebral cage 100 is positioned. As also seen in FIG. 1A, the body 102 can define a vertical axis 108 extending perpendicular to the longitudinal axis 104 and between the top 120 and the bottom 122 of the body 102. As further seen in FIG. 1A, the body 102 can define a lateral axis 106 extending perpendicular to both the longitudinal axis 104 and the vertical axis 108.

As seen in FIGS. 1A and 1B and 2A and 2B, the combination of the segments 116 and the living hinges 118 allow deployment of the body 102 of the intervertebral cage 100, which deployment decreases the distance between the proximal end 110 and the distal end 112 and increases the width of the body 102 as measured along the lateral axis 106.

The segments 116 and the living hinges 118 can comprise a variety of shapes and sizes. In some embodiments, for example, the shapes and sizes of the segments 116 and/or the living hinges 118 can be determined by the desired size of the intervertebral cage 100, the desired deployment force, the desired expanded resulting shape, the desired unexpanded shape, and/or a number of other considerations.

The body 102 may be manufactured using known additive manufacturing or 3D printing methods. For example, body 102 may be constructed layer-by-layer, bottom-to-top, from a biocompatible material such that the segments 116 are integrally connected by living hinges 118. The body 102 can be manufactured in a single integral piece that cannot be taken apart without dividing the body. The single piece can be manufactured by 3D printing process. Similar fabrication processes are known by the names: additive manufacturing, rapid manufacturing, layered manufacturing, rapid prototyping, laser sintering, electron beam melting (EBM), etc. All of these fabrication processes use a similar operating principle of scanning an energized beam over a bath of material to solidify a precise pattern of the material to form each layer until the entire component is complete. The present invention m

In some embodiments, an intervertebral cage 100 can be configured such that dimensions of the intervertebral cage 100 vary along one, two, or three of the above discussed axes 104, 106, 108 when the intervertebral cage 100 is moved from an unexpanded configuration to an expanded configuration.

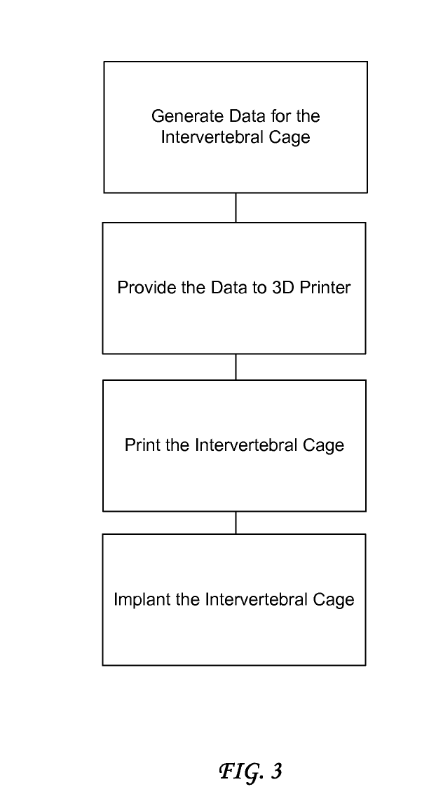

FIG. 3 shows one embodiment of a 3D printing process flowchart. The data used in manufacturing the cage may use predefined data of standard cage sizes or use imaging data taken of the patient undergoing the surgical procedure. The data may be from a software program generating a geometric representation of a 3D physical model in a data format supported by a 3D printer, and sending the geometric representation to a 3D printer to create a 3D physical model.

3D printers require a geometric representation of an object in order to fabricate the geometric shapes required in making a 3D physical model. Typical geometric representation of an object may include one or a combination of the following forms: a list of 3D points for the entire body of the object with locational and material information defined at each 3D point, a group of 3D contours to define the shape of the object on each image plane, or surface models consisting of triangles or polygons or surface patches delineating the body of the object.

A 3D physical model of the cage may have one or more pieces and one or multiple colors, and may be made of one or multiple materials. The conversion process from input image data set to a geometric representation understood by a 3D printer may be either dependent on or independent of imaging modality or any other image information. The process may be implemented as a software program on a computer, a computer processing board, or the controller board of a 3D printer. It may be implemented as but not limited to: a program script file with processing instructions and parameters, a binary executable program with processing instructions and parameters, a dynamically linked library (DLL), an application plug-in, or a printer device driver. A printing data program may be loaded locally on a 3D printer computer or reside on a remote server connected through a computer network.

Once the data is received, the 3D printer prints the cage and the cage may then be sent to surgery to be implanted in the patient.

The Variable Volume Pouch

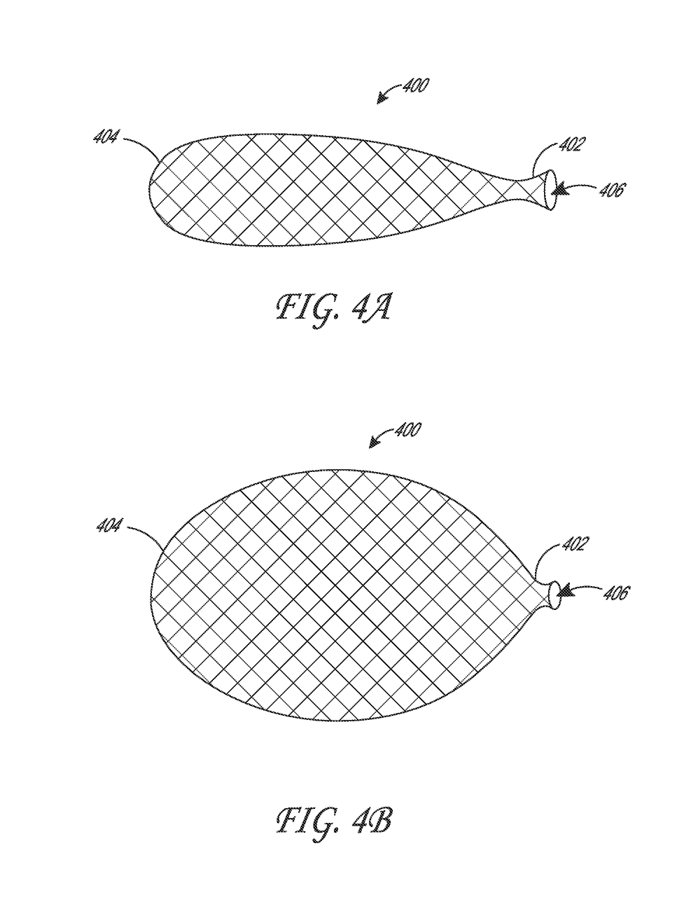

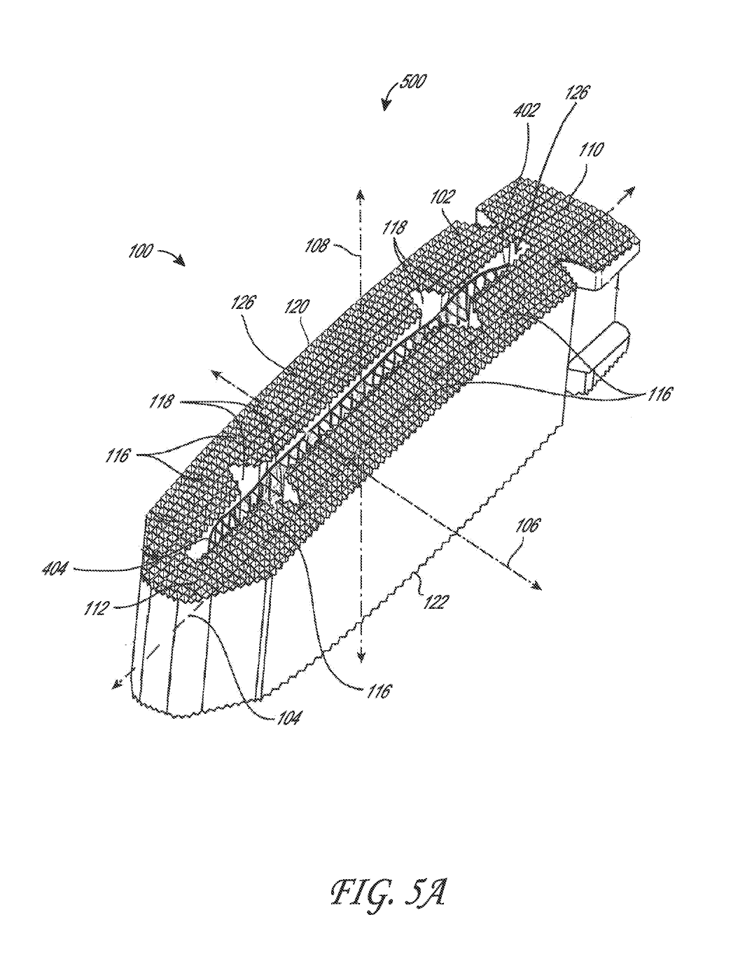

Some embodiments of an intervertebral cage apparatus can include a variable volume pouch. FIG. 4A depicts a perspective view of one embodiment of a variable volume pouch 400 in an unexpanded state and FIG. 4B depicts one embodiment of a variable volume pouch 400 in an expanded state. The variable volume pouch 400 can be configured for expansion in response to receiving material in an internal portion of the variable volume pouch 400. In some embodiments, the variable volume pouch 400 can be configured to resist compressive forces when the variable volume pouch 400 is filled with material. One example of a variable volume pouch is the OptiMesh® Deployable Grafting System available from Spineology, Inc. Although some details of the variable volume pouch 400 and methods of use are provided herein, further details can be found in U.S. Pat. No. 5,549,679 published on Mar. 1, 1995, entitled “Expandable Fabric Implant For Stabilizing the Spinal Motion Segment,” and in U.S. Pat. No. 5,571,189 published on Nov. 5, 1996, entitled “Expandable Fabric Implant For Stabilizing the Spinal Motion Segment,” both of which are incorporated herein in their entirety by reference.

The variable volume pouch can comprise a variety of shapes and sizes. In some embodiments, for example, the variable volume pouch 400 can be shaped to allow uniform expansion of the variable volume pouch 400 when material is added into the internal portion of the variable volume pouch 400. In some embodiments, for example, the variable volume pouch can be approximately spherical, ovular, elongate, cylindrical, rectangular, or have any other desired shape. In the embodiment depicted in FIGS. 4A and 4B, the variable volume pouch 400 is approximately balloon shaped. As also seen in FIGS. 4A and 4B, the variable volume pouch 400 comprises a first end 402 and a second end 404 positioned opposite the first end 402. As seen in FIGS. 4A and 4B, the variable volume pouch 400 further comprises a single opening 406 located at the first end 402.

In some embodiments, the variable volume pouch 400 can include features configured to allow the selectable sealing and/or closing of the opening 406. These features can include, for example, one or several ties, one or several drawstrings, one or several plugs, or any other mechanical or other feature configured to allow the sealing and/or closing of the opening 406.

The variable volume pouch 400 can comprise a variety of materials. In some embodiments, the variable volume pouch can comprise a natural material, a synthetic material, a man-made material, a polymer, composite material, an elastic material, an inelastic material and/or any other desired material. In some embodiments, and as depicted in FIGS. 4A and 4B, the variable volume pouch 400 can comprise a woven material. Advantageously, a woven material can allow expansion of the variable volume pouch 400 to a desired maximum size.

The variable volume pouch 400 can comprise a variety of sizes. In some embodiments, the variable volume pouch 400 can be sized to allow placement between two vertebrae. Specifically, in some embodiments, the variable volume pouch 400 can be sized to fit between two vertebrae and specifically between the end plates of two vertebrae.

The Intervertebral Cage Apparatus

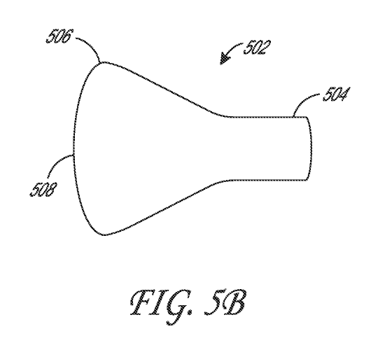

FIG. 5A depicts a perspective view of one embodiment of an intervertebral cage apparatus 500. The intervertebral cage apparatus 500 comprises the intervertebral cage 100 and the variable volume pouch 400 located within the internal volume 126 of the intervertebral cage 100. In some embodiments, the variable volume pouch 400 can be affixed to all or portions of the intervertebral cage 100. In some embodiments, for example, the variable volume pouch 400 can be inserted into the internal volume 126 of the intervertebral cage 100 such that the second end 404 of the variable volume pouch 400 is proximate to the distal end 112 of the intervertebral cage 100 and the first end 402 is proximate to the proximal end 110 of the intervertebral cage 100. In some advantageous embodiments, in which the first end 402 is proximate to the proximal end 110 of the intervertebral cage 100, the opening 406 of the variable volume pouch 400 is located proximate to the proximate aperture 124 of the body 102 of the intervertebral cage 100. Thus, in some embodiments, the variable volume pouch 400 can be inserted into the internal volume 126 of the body 102 of the intervertebral cage 100 through the proximal aperture 124. In such an embodiment, after the variable volume pouch 400 is inserted into the internal volume 126 of the body 102 via the proximal aperture 124, the variable volume pouch 400 can be partially or completely affixed to the body 102 of the intervertebral cage 100. In some embodiments, the variable volume pouch 400 can be affixed to the body 102 of the intervertebral cage 100 such that the expansion of the variable volume pouch 400 can result in the deployment of the body 102 of the intervertebral cage 100 and in some embodiments, the affixation of the variable volume pouch 400 to the body 102 of the intervertebral cage 100 can result in the expansion of the variable volume pouch 400 when the body 102 of the intervertebral cage 100 is expanded.

In some embodiments, the intervertebral cage apparatus 500 can further comprise a plug 502. The plug 502 can be configured to sealingly fit within the proximal aperture 124 to seal the proximal aperture, to secure the first end 402 of the variable volume pouch 400 to the proximal end 110 of the intervertebral cage 100, and to seal the opening 406 of the variable volume pouch. In some embodiments, the plug 502 can be further configured to facilitate in the deployment of the intervertebral cage 100. The plug 502 can comprise a variety of shapes and sizes, and can be made from a variety of materials, including, for example, all of the materials from which the intervertebral cage 100 can be made.

In some embodiments, the plug 502 can comprise a proximal shaft 504 and a distal head 506. The proximal shaft 504 can comprise a variety of shapes and sizes. In some embodiments, the proximal shaft 504 can be sized and shaped to seal the proximal aperture 124, and specifically can be sized and shaped with larger dimensions than the proximal aperture 124. In some embodiments, the configuration of the proximal shaft 504 with dimensions larger than the dimensions of the proximal aperture 124 can facilitate the retention of the plug 502 in the proximal aperture 124.

In some embodiments, the distal head 506 can comprise a variety of shapes and sizes. In some embodiments, the distal head 506 can be conical shaped, having a distal base 508, and extending towards the apex in the direction of the proximal shaft 504. The distal head 506 can be shaped, in some embodiments, to facilitate in deploying the intervertebral cage 100.

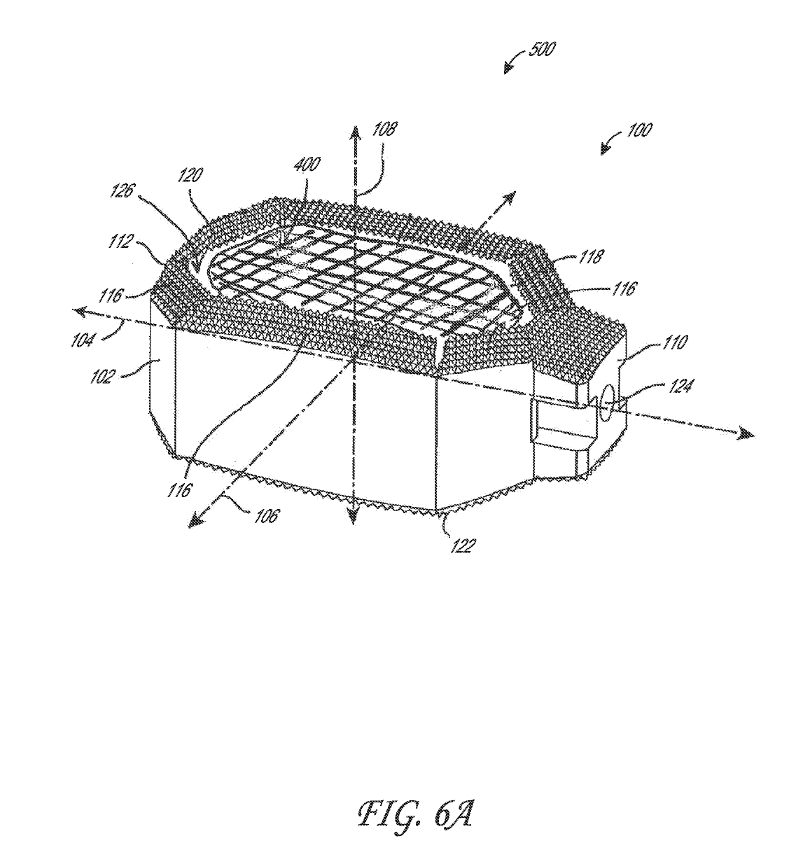

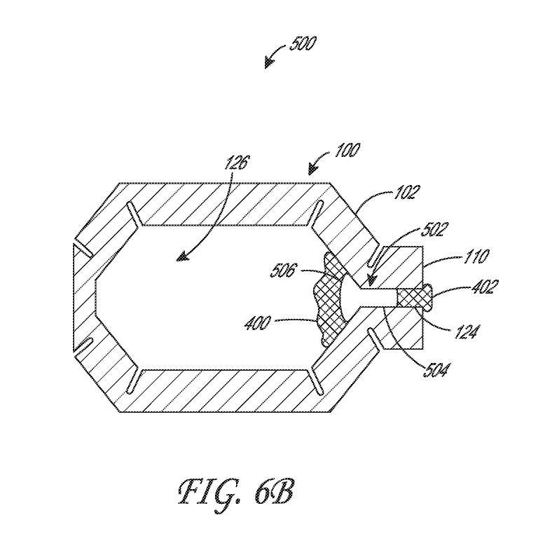

FIG. 6A depicts one embodiment of the intervertebral cage apparatus 500 in an expanded configuration in which the body 102 of the intervertebral cage 100 is expanded and in which the variable volume pouch 400 is in its expanded configuration. As seen in FIG. 6A, the variable volume pouch 400 in its expanded configuration fills and/or substantially fills the internal volume 126 of the intervertebral cage 100.

FIG. 6B is a cutaway top-view of the intervertebral cage apparatus 500 in an expanded configuration. As seen in FIG. 6B, the proximal shaft 504 of the plug 502 is located in the proximal aperture 124 of the body 102 of the intervertebral cage 100. As also seen in FIG. 6B, the proximal shaft 504 of the plug has expanded the diameter of the proximal aperture 124, and is thereby secured within the proximal aperture 124. As also seen in FIG. 6B, the plug 502 is positioned within the proximal aperture 124 such that a portion of the first end 402 of the variable volume pouch 400 is between the proximal shaft 504 and the wall of the proximal aperture, thereby securing the variable volume pouch 400.

FIG. 6B further depicts the distal head 506 of the plug 502 extending into the internal volume 126 of the intervertebral cage 100. As seen in FIG. 6B, the distal head 506 is engaging portion of the body 102, to thereby bias the body 102 of the intervertebral cage 100 towards an expanded configuration.

In some embodiments, in which the plug 502 is used in connection with the intervertebral cage apparatus 500, the variable volume pouch 400 can be inserted into the intervertebral cage 100 through the proximal aperture 124 and positioned such that the first end 402 of the variable volume pouch 400 and the opening 406 are proximate to the proximal aperture 124. In some embodiments, the variable volume pouch 400 can be at least partially affixed to the intervertebral cage 100. After the variable volume pouch 400 is inserted into the intervertebral cage 100, positioned, and if desired, at least partially affixed to the intervertebral cage 100, the variable volume pouch can be filled and/or the intervertebral cage 100 can be expanded.

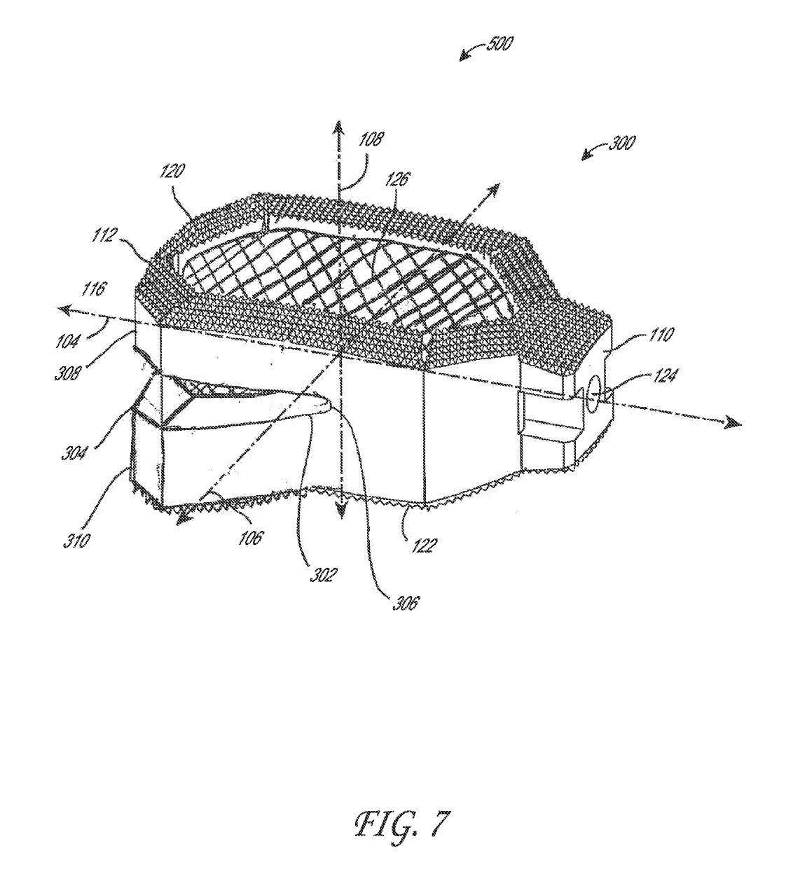

FIG. 7 depicts an alternative embodiment of the intervertebral cage apparatus 500. Specifically, FIG. 7 depicts an embodiment of the intervertebral cage apparatus 500 comprising a variable volume pouch 400 shown in this figure in its expanded state, and the intervertebral cage 300 comprising a lateral split 302 shown in its fully expanded configuration. As seen in FIG. 7, the intervertebral cage 300 is expanded in both the lateral direction 106 as measured along the lateral axis 106 and expanded in the vertical direction as measured along the vertical axis 108. As seen in FIG. 7, the variable volume pouch 400 substantially fills and/or fills the internal volume 126 of the intervertebral cage 300.

The Deployment System

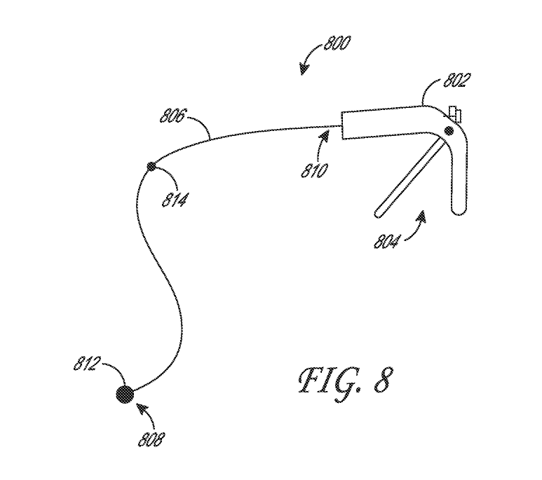

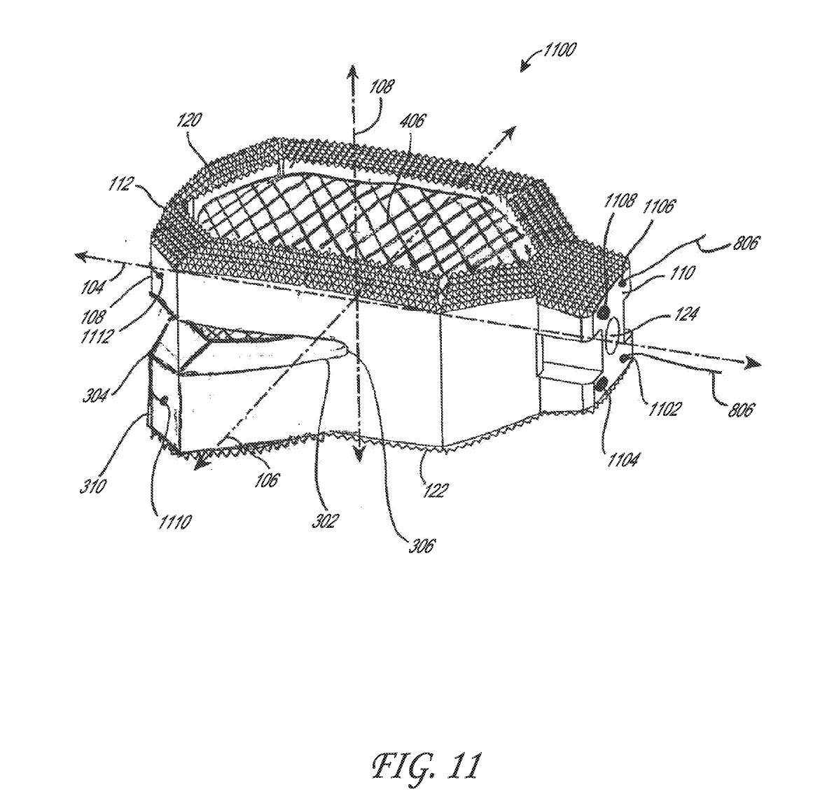

Some embodiments relate to systems and devices for the insertion and deployment of an intervertebral cage apparatus 500 and/or of the intervertebral cage 100, 300 (described further in respect to FIG. 11). FIG. 8 depicts one embodiment of insertion deployment system 800. As seen in FIG. 8, the deployment system 800 can include a deployment tool 802. The deployment tool 802 can be configured to facilitate in the insertion of the intervertebral cage apparatus 500 and/or the intervertebral cage 100, 300 and to control the deployment of the intervertebral cage apparatus 500 and/or the intervertebral cage 100, 300.

The deployment tool 802 can comprise a variety of shapes and sizes and can comprise a variety of features. In some embodiments, for example, the deployment tool 802 can be a mechanical device, an electromechanical device and/or an electrical device. In some embodiments, for example, the deployment tool 802 can be manually operated, can be electrically controlled, and/or can be controlled using any other desired control technique. As depicted in FIG. 8, the deployment tool 802 comprises a control interface 804. The control interface 804 can be configured to allow a user to control the deployment tool 802 and the insertion and/or deployment of the intervertebral cage apparatus 500 and/or the intervertebral cage 100, 300. In some embodiments, for example, the control interface can comprise any feature, system, and/or module configured to receive user input and use that input to effect the deployment of the intervertebral cage apparatus 500 and/or the intervertebral cage 100, 300. As depicted in FIG. 8, the control interface 804 can comprise a simple manual control configured to apply a force to one end of a deployment cable 806.

In some embodiments in which the deployment tool 802 can be used in connection with other features to insert the intervertebral cage 100, 300. In such embodiments, the deployment tool 802 can be used with a rigid shaft. In one embodiment, the rigid shaft can comprise a proximal end that is affixed to the deployment tool 802 and a distal end configured to engage with the intervertebral cage 100, 300. In some embodiments, these features configured to engage with the intervertebral cage 100, 300 and located at the distal end of the rigid shaft can comprise one or several prongs (not shown) configured to engage portions of the intervertebral cage 100, 300. In some embodiments, the features configured to selectively affix the intervertebral cage 100, 300 to the deployment tool 802, can allow the manipulation and movement of the intervertebral cage 100, 300 along and/or about any of the axes 104, 106, 108 of the intervertebral cage 100, 300.

In some embodiments, the rigid shaft can be configured to allow the passage of the deployment cable 806 from the deployment tool 802 to the intervertebral cage 100, 300. In some embodiments, the deployment cable 806 can pass along the rigid shaft and/or through the rigid shaft from the deployment tool 802 to the intervertebral cage 100, 300. The passing of the deployment cable 806 from the deployment tool 802 to the intervertebral cage 100, 300 can be facilitated by one or several channels located within the rigid shaft. In some embodiments, these rigid channels can be located on an exterior surface of the rigid shaft, and or located within the rigid shaft. In some embodiments, the channels can extend the entire length of the rigid shaft, and/or along portions of the rigid shaft.

In some embodiments in which the deployment tool 802 is only used for deployment of the intervertebral cage 100, 300 a separate insertion tool and/or tools can be used in the insertion of the intervertebral cage 100, 300. Some embodiments of such an insertion tool and/or implantation tool can be found in U.S. Publication No. 2012/0083887 published on Apr. 5, 2012 which is incorporated herein in its entirety by reference.

The deployment cable 806 can be configured to transfer a force from the deployment tool 802 to the intervertebral cage apparatus 500 and/or the intervertebral cage 100, 300. In some embodiments, the deployment cable 806 can be configured to facilitate the deployment of the intervertebral cage apparatus 500 and/or the intervertebral cage 100, 300 and/or to facilitate in maintaining the intervertebral cage apparatus 500 and/or the intervertebral cage 100, 300 in an expanded configuration. In some embodiments, the deployment cable 806 can be configured for use as a marker, and specifically, can be used as a marker to indicate the position of the intervertebral cage 100, 300 and/or to determine whether and to what extent the intervertebral cage 100, 300 has been expanded. In some embodiments, for example, the deployment cable 806 can include regularly spaced features that can allow determination of whether and/or to what extent the intervertebral cage 100, 300 is expanded by allowing the determination of the length of the deployment cable 806 within the intervertebral cage 100, 300 As the deployment of the intervertebral cage 100, 300 may, in some embodiments, change a dimension of the intervertebral cage 100, 300 the determination of the length of the portion of the deployment cable 806 located within the intervertebral cage can facilitate in determining whether and/or to what extent the intervertebral cage 100, 300 is expanded.

The deployment cable 806 can comprise a variety of shapes and sizes and can be made from a variety of materials. All or part of the deployment tool may be manufactured by 3D printing. In some embodiments, the deployment cable 806 can comprise any shape and size and can be made from any material capable of applying and withstanding the forces necessary to deploy the intervertebral cage apparatus 500 and/or the intervertebral cage 100, 300.

As depicted in FIG. 8, the deployment cable 806 comprises a first end 808 and a second end 810. In some embodiments, the first end 808 can comprise an attachment feature 812. The attachment feature 812 can be any feature configured to allow the attachment of the deployment cable 806 to a portion of the intervertebral cage 100, 300 and/or to prevent the movement of the deployment cable 806 in one or several specified directions relative to the intervertebral cage 100, 300.

The attachment feature 812 can comprise a variety of shapes and sizes and can be made from a variety of materials. In one embodiment, for example, the attachment feature 812 can comprise a shape and/or size that allows the attachment feature 812 to engage a portion of the intervertebral cage 100, 300 and thereby restrict the movement of the deployment cable 806 relative to the intervertebral cage 100, 300. As specifically seen in FIG. 8, in some embodiments, the attachment feature can comprise a spherical feature located at the first end 808 of the deployment cable 806.

In some embodiments, the deployment cable 806 can comprise a breakage point (not shown). In some embodiments, the breakage point can be a portion of the deployment cable 806 that is configured to sever, break, and/or separate when a force threshold is exceeded. In some embodiments, the force threshold for the breakage point can be below the force threshold that would cause other portions and/or features such as, for example, the attachment feature 812 and/or the locking feature 814 of the deployment cable 806 to break or fail. In some embodiments, the breakage point can be positioned between, for example, between the locking feature 814 and the second end 810 of the deployment cable 806. Advantageously, as the application of a force above the force threshold results in the breakage of the deployment cable 806 at the breakage point, such positioning of the breakage point can eliminate the need to cut the deployment cable 806 after the intervertebral cage 100, 300 has been expanded.

As also seen in FIG. 8, the second end 810 of the deployment cable 806 can be connected to a portion of the deployment tool 802. As further seen in FIG. 8, in some embodiments, the deployment cable 806 further comprises a locking feature 814 that can be, for example, located at any position along the deployment cable, and in some embodiments, located between the attachment feature 812 and the second end 810 of the deployment cable 806. The locking feature 814 can be configured to allow a user to lock and/or secure the intervertebral cage 100, 300 in an expanded configuration. In some embodiments, the locking feature 814 can comprise the size and/or shape configured to interact with a portion of the intervertebral cage 100, 300 and thereby prevent the intervertebral cage 100, 300 from returning to an unexpanded configuration after the intervertebral cage 100, 300 has been expanded.

In some embodiments, for example, the distance between the attachment feature 812 and the locking feature 814 can vary. Specifically, for example, the distance between the attachment feature 812 and the locking feature 814 can vary based on the size of the intervertebral cage 100, 300, the distance that the deployment cable 806 must be moved before the intervertebral cage 100, 300 deploys, and/or any other desired parameters.

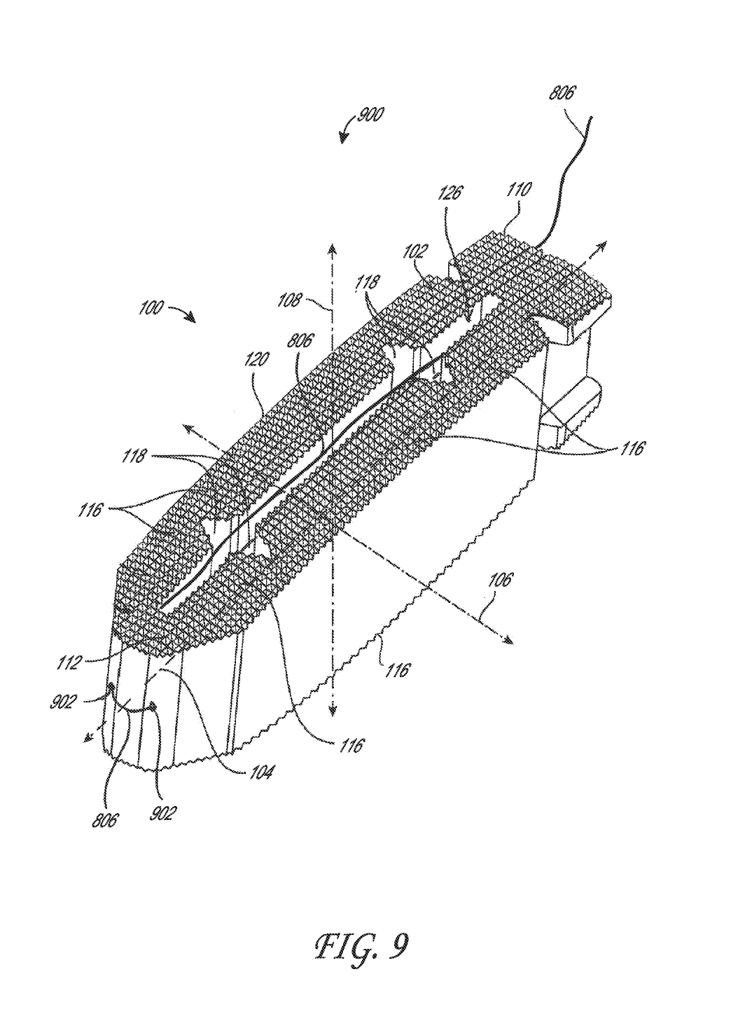

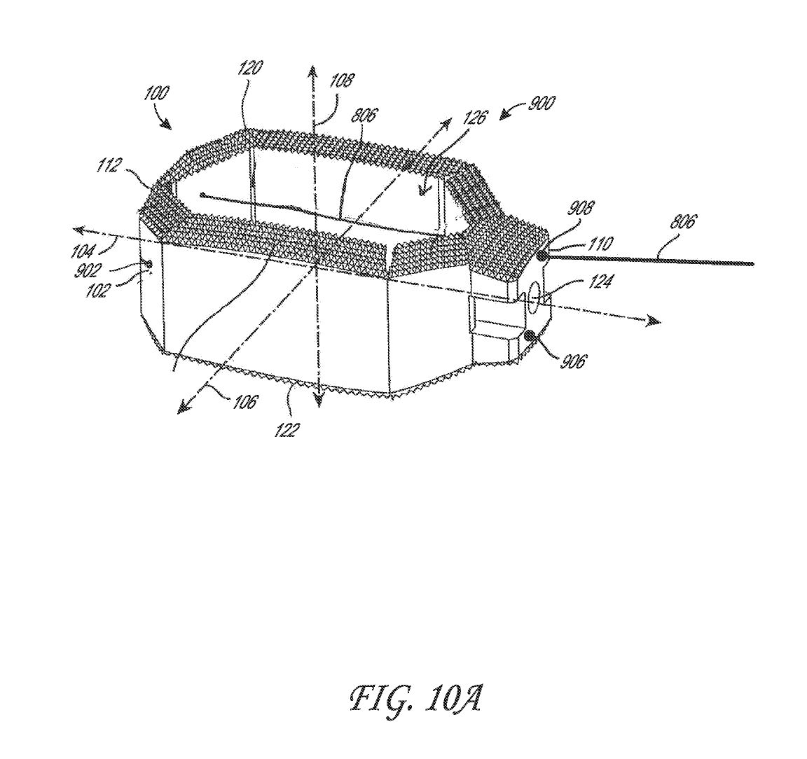

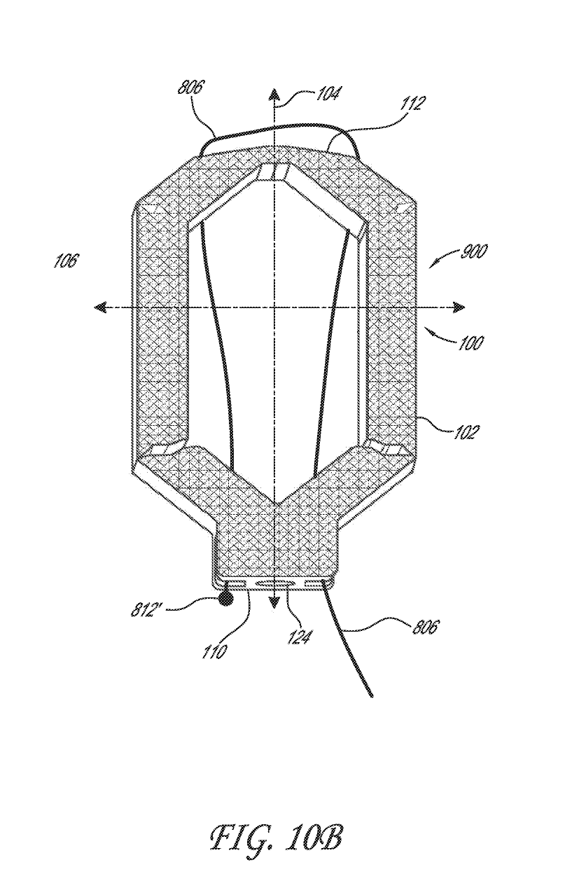

FIGS. 9, 10A and 10B depict perspective views of one embodiment of an intervertebral cage apparatus 900. Specifically, FIG. 9 depicts one embodiment of the intervertebral cage apparatus 900 in an unexpanded configuration and FIGS. 10A and 10B depict a perspective view of one embodiment of an intervertebral cage apparatus 900 in an expanded configuration. As seen in FIGS. 9, 10A and 10B, the intervertebral cage 100 can be configured for use with a deployment cable 806. In some embodiments, for example, the intervertebral cage 100 can comprises one or several opening and/or one or several channels configured to receive, direct, and/or hold a portion of the deployment cable. These openings can comprise a variety of shapes and sizes, and can be located on any desired portion of the intervertebral cage. In some embodiments, the size and shape of the openings can be determined by the size and shape of features accommodated by the openings, such as, for example, the deployment cable 806, the attachment feature 812, and/or the locking feature 814. Specifically, for example, the intervertebral cage 100 can comprise one or several distal openings 902 located proximate to the distal end 112 of the body 102 of the intervertebral cage 100 and a first and/or second proximal opening 906, 908 located proximate to the proximal end 110 of the intervertebral cage 100.

The distal opening(s) 902, the first proximal opening 906, and the second proximal opening 908 can be configured to receive a portion of the deployment cable 806. In some embodiments, for example, all or some of the distal opening(s) 902, the first proximal opening 906 and/or the second proximal opening 908 can guide the deployment cable 806 into and out of a portion of the intervertebral cage 100. In some embodiments, these openings 902, 906, 908 can be connected to one or several channels that pass through all or portions of the intervertebral cage 100. Thus, in some embodiments, the deployment cable may enter into the intervertebral cage 100 through the first proximal opening 906, and after passing through all or a portion of the intervertebral cage 100, the deployment cable 806 may then exit the channel inside the intervertebral cage 100 via another opening such as, for example, the distal opening 902 and/or the second proximal opening 908.

As further seen in FIG. 9, in some embodiments, the deployment cable 806 can pass through the internal volume 126 of the body 102 of the intervertebral cage 100. Specifically, in some embodiments, all or portions of the deployment cable 806 can extend from a first proximal opening 906 to a distal opening 902 and/or from a distal opening 902 to a second proximal opening 908 alongside an inner surface of the cage 100.

In some embodiments, a plurality of deployment cables 806 can be used in connection with a single intervertebral cage 100, 300. In some embodiments, the number of deployment cables 806 can be determined by the desired type of deployment. Thus, in some embodiments, the more deployment cables 806 may be used to achieve a more complex deployment motion.

FIG. 11 depicts one embodiment using a plurality of deployment cables. Specifically, FIG. 11 depicts a further embodiment of an intervertebral cage apparatus 1100. As seen in FIG. 11, the intervertebral cage apparatus 1100 comprises an intervertebral cage 300 comprising a body 102 having a proximal end 110 and a distal end 112. The body 102 defines a longitudinal axis 104 extending down the center of the body 102 and between the proximal end 110 and the distal end 112. The body 102 of the intervertebral cage 300 further comprises a plurality of segments 116 joined by living hinges 118 define an internal volume 126 of the body and a top 120 and a bottom 122 and defines a vertical axis 108 extending between the lop 120 and the bottom 122 and perpendicular to the longitudinal axis 104. The body 102 of the intervertebral cage 300 further defines a lateral axis 106 which extends perpendicular to both the longitudinal axis 104 and the vertical axis 108.

As also seen in FIG. 11, the body 102 of the intervertebral cage 300 comprises a lateral split 302. The lateral split 302 can be configured to allow the expansion of the body 102 of the intervertebral cage 300. In some embodiments, for example, the lateral split 302 can be configured to allow the expansion of all or a portion of the body 102 of the intervertebral cage 300 in a direction perpendicular to the lateral split 302. The lateral split 302 comprises a first end 304 and a second end 306. The first end 304 of the lateral split 302 is located proximate to the distal end 112 of the body 102 and the second end 306 of the lateral split 302 is located approximately in the middle of the body 102. The lateral split 302 divides the body 102 at least partially into a top portion 308 and a bottom portion 310. Advantageously, the division of the body 102 into a top portion 308 and into a bottom portion 310 by a lateral split 302 allows the expansion of the body 102 of the intervertebral cage 300. In some embodiments, for example, this expansion of the body 102 of the intervertebral cage 300 can be perpendicular to the lateral split 302, and in some embodiments, this expansion of the body 102 can be nonperpendicular to the lateral split 302. The top portion 308 and the bottom portion 310 of the body 102 allow the expansion of the body 102 in a direction parallel to the lateral axis 106 by the expansion of the lateral split 302.

As further seen in FIG. 11, the intervertebral cage 300 further comprises a lower first proximal opening 1102, a lower second proximal opening 1104, an upper first proximal opening 1106, an upper second proximal opening 1108, a lower distal opening 1110, and an upper distal opening 1112.

As also seen in FIG. 11, the intervertebral cage apparatus 1100 comprises two deployment cables 806. One of the deployment cables 806 depicted in FIG. 11 inserts through the lower first proximal opening 1102 and then passes through the variable volume pouch 400 where it exits through one of at least one lower distal opening 1110 before again passing through the variable volume pouch and to the lower second proximal opening 1104. This path of the deployment cable 806 secures a portion of the variable volume pouch 400 to the intervertebral cage 300 and specifically to the bottom portion 310 of the intervertebral cage 300.

As also seen in FIG. 11, the other deployment cable 806 passes through the upper first proximal opening 1106 and then through the variable volume pouch to at least one of the upper distal openings 1112 before again passing through the variable volume pouch and to the upper second proximal opening 1108. Similar to the deployment cable 806 passing through the lower openings 1102, 1104, 1110, the deployment cable 806 passing through the upper openings 1106, 1108, 1112, secures a portion of the variable volume pouch 400 to the intervertebral cage 300 and specifically to the top portion 308 of the intervertebral cage 300. Advantageously, the securement of the variable volume pouch 400 to the top portion 308 and the bottom portion 310 allows use of a variable volume pouch 400 to at least partially vertically deploy the intervertebral cage 300 with respect to the vertical axis 108 by filling the internal portion of the variable volume pouch 400.

While FIG. 11 depicts an embodiment in which two deployment cables 806 are used and showing specific positions for the openings 1102, 1104, 1106, 1108, 1110, 1112, a person of skill in the art will recognize that any number of deployment cables 806 can be used in connection with the intervertebral cage 300 and that a wide variety of positions for the openings can be used.

Methods of Using an Intervertebral Cage Apparatus

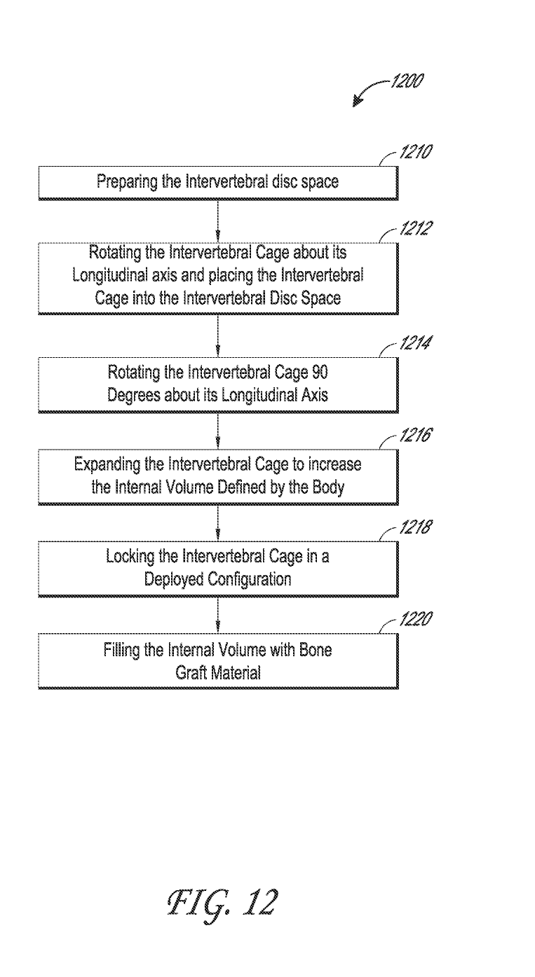

FIG. 12 is a flowchart illustrating one embodiment of process 1200 for using the intervertebral cage 100, 300 and/or the intervertebral cage apparatus 500, 1100. The process begins at block 1210 wherein the intervertebral disc space is prepared, for example, by removing a portion of the annulus, evacuating the nucleus, and then removing the cartilaginous endplates.

After the intervertebral disc space is prepared, the process 1200 proceeds to block 1212 wherein the intervertebral cage 100, 300 is placed into the intervertebral disc space. In one embodiment, the intervertebral cage 100, 300 is rotated about its longitudinal axis 104 and placed in the intervertebral disc space such that the vertical axis 108 of the body 102 of the intervertebral cage 100, 300 is parallel to the vertebral endplates.

The process 1200 proceeds to block 1214 wherein the intervertebral cage 100, 300 is rotated 90 degrees about its longitudinal axis 104. After the rotation of the intervertebral cage 100, 300, the top 120 and the bottom 122 contact the vertebral endplates. In some embodiments, in which the distance between the top 120 and the bottom 122 of the body 102 of the intervertebral cage 100, 300 is larger than the width of the body 102 of the intervertebral cage 100, 300 as measured parallel to the lateral axis 106, the 90 degree rotation of the body 102 along its longitudinal axis 104 increases the height of the intervertebral disc space.

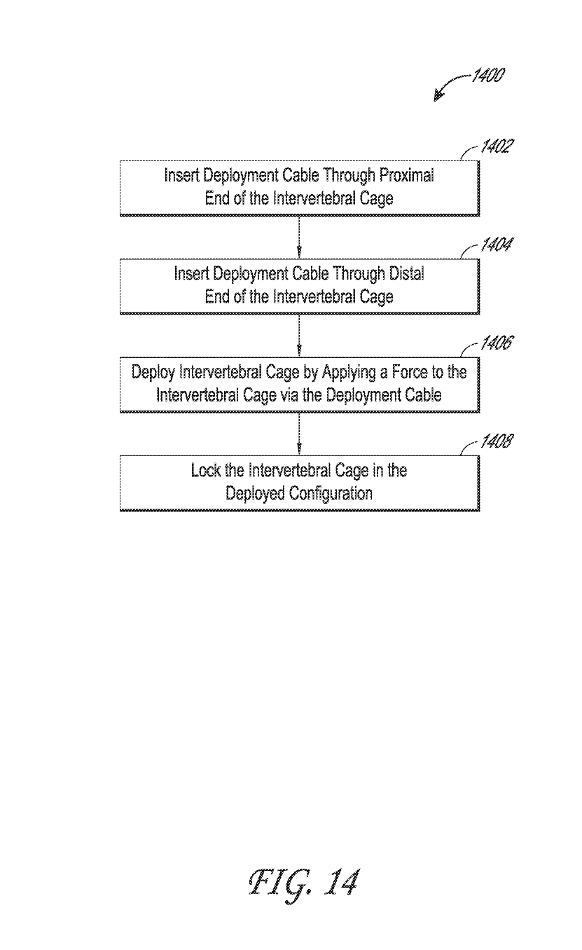

After the intervertebral cage 100, 300 is rotated 90 degrees about its longitudinal axis 104, the process 1200 proceeds to block 1216 wherein the intervertebral cage 100, 300 is expanded to increase the internal volume 126 defined by the body 102, and in some embodiments, defined by the segments 116 and living hinges 118 forming the body 102. In some embodiments, the expansion of the intervertebral cage 100, 300 can proceed as outlined in step 1406 as depicted in FIG. 14.

In some embodiments, the body 102 is expanded until the body 102 attains an expanded configuration. In some embodiments, for example, in which a the intervertebral cage 100, 300 is used in connection with a deployment tool 802, the actuation of the deployment tool 802 can cause the deployment of the intervertebral cage 100, 300 and thereby the expansion of the intervertebral cage 100, 300 and the expansion of the internal volume 126 of the intervertebral cage 100, 300. In some embodiments in which the intervertebral cage 100, 300 comprises a body 102 made of a memory material such as, for example, PEEK Altera™, the intervertebral cage 100, 300 can be expanded by triggering the memory material such that the intervertebral cage 100, 300 transforms from the unexpanded, second position to the expanded, first position. In some embodiments, triggering can be temperature induced, stress induced, electrically and/or mechanically induced, chemically induced, and/or through any other triggering mechanism. In some specific embodiments, the triggering can be induced when a threshold temperature of the intervertebral cage 100, 300 is exceeded, or when a stress threshold for the intervertebral cage 100, 300 is surpassed.

After the intervertebral device is expanded to increase the internal volume 126 defined by the body 102, the process 1200 can, in some embodiments, proceed to block 1218 wherein the intervertebral device is locked in a expanded configuration with a locking mechanism such as, for example, a deployment cable 806. Although the process 1200 can, in some embodiments, include block 1218, the steps of this block can be omitted and the process 1200 can proceed to block 1220.

The process 1200 can then proceed to block 1220, wherein the internal volume 126 of the body 102 of the intervertebral cage 100, 300 is filled with bone graft material to permit bone fusion between adjacent vertebrae. In some embodiments, the internal volume 126 of the body 102 of the intervertebral cage 100, 300 can be filled via the proximal aperture 124 located in the proximal end 110 of the body 102 of the intervertebral cage 100, 300.

A person of skill in the art will recognize that the steps of the aforementioned process can be performed in the same order, or in a different order. A person of skill in the art will further recognize that the process 1200 can include more or fewer steps than those outlined above.

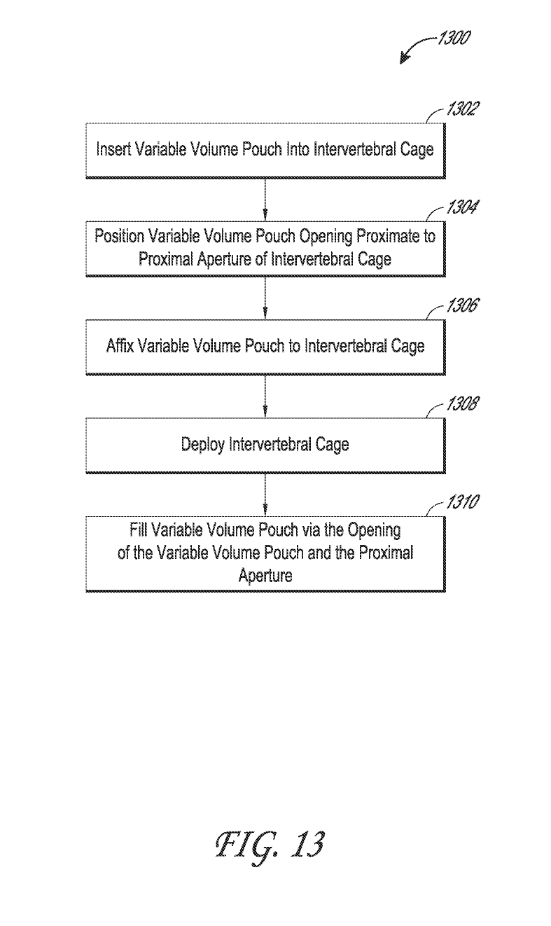

FIG. 13 is a flow chart illustrating one embodiment of the process 1300 for using an intervertebral cage apparatus 500. In some embodiments, parts of the process 1300 can be performed before insertion of the intervertebral cage apparatus 500 into an intervertebral space, and in some embodiments, parts of the process 1300 can be performed after insertion of the intervertebral cage apparatus into an intervertebral space.

The process 1300 begins at block 1302 wherein the variable volume pouch 400 is inserted into the intervertebral cage 100, 300. In some embodiments, for example, the insertion of the variable volume pouch 400 into the intervertebral cage 100, 300 can be performed using a variety of tools and techniques. In some embodiments, for example, the variable volume pouch can be inserted into the intervertebral cage 100, 300 via the proximal aperture 124 in the proximal end 110 of the body 102 of the intervertebral cage 100, 300. In some embodiments, the variable volume pouch 400 can be inserted into the internal volume 126 of the intervertebral cage 100, 300 via the proximal aperture 124 located in the proximal end 110 of the intervertebral cage 100, 300. In some embodiments, the variable volume pouch 400 can be pre-inserted into the intervertebral cage 100, 300, and the process 1300 can begin at a block other than block 1302.

After the variable volume pouch 400 has been inserted into the intervertebral cage 100, 300, the process 1300 then proceeds to block 1304 wherein the opening 406 of the variable volume pouch 400 is positioned proximate to the proximal aperture 124 of the intervertebral cage 100, 300. In some embodiments, the positioning of the opening 406 of the variable volume pouch 400 proximate to the proximal aperture 124 of the intervertebral cage 100, 300 can be achieved, for example, by inserting the second end 404 of the variable volume pouch 400 through the proximal aperture 124 before inserting the first end 402 of the variable volume pouch 400 through the proximal aperture 124. By following this insertion procedure, and thereby inserting the second end 404 of the variable volume pouch 400 through the proximal aperture 124 first, the opening 406 of the variable volume pouch 400 which is located at the first end 402 of the variable volume pouch 400 can be easily positioned proximate to the proximal aperture 124 of the intervertebral cage 100, 300. In some embodiments in which the variable volume pouch 400 is pre-inserted into the intervertebral cage 100, 300, the process 1300 can begin at block 1304. In some embodiments, the opening 406 of the variable volume pouch 400 can be pre-positioned proximate to the proximal aperture 124 of the intervertebral cage 100, 300, and the process 1300 can begin at a block other than block 1304.

After the opening 406 of the variable volume pouch 400 has been positioned proximate to the proximal aperture 124 of the intervertebral cage 100, 300, the process 1300 proceeds to block 1306 wherein the variable volume pouch 400 is affixed to the intervertebral cage 100, 300. In some embodiments, for example, the variable volume pouch 400 can be affixed to all or portions of the intervertebral cage 100, 300 and specifically to all or portions of the body 102 of the intervertebral cage 100, 300. In some embodiments, for example, the variable volume pouch 400 can be affixed to the body 102 of the intervertebral cage 100, 300 along the portions of the body 102 defining the internal volume 126. Specifically, portions of the variable volume pouch 400 can be affixed to the segments 116 and living hinges 118 that constitute the body 102.

In some embodiments, the variable volume pouch 400 can be affixed to the body 102 of the intervertebral cage 100, 300 with features located on the body 102 of the intervertebral cage 100, 300 such as, for example, one or several fasteners, one or several hooks, one or several snaps, one or several adhesive regions, and/or any other desired feature located on either or both of the variable volume pouch 400 and the body 102 of the intervertebral cage 100, 300. In some embodiments, for example, the variable volume pouch 400 can be affixed to the intervertebral cage 100, 300 through additional features that are not an integral part of either the variable volume pouch 400 or the body 102 of the intervertebral cage 100, 300. In some embodiments, these features can include, for example, one or several deployment cables 806. In some embodiments, for example, the deployment cable 806 can be fused to affix the variable volume pouch 400 to the intervertebral cage 100, 300. In some specific embodiments, the deployment cable 806 can be inserted through a portion of the intervertebral cage 100, 300 such as, for example, the body 102, be threaded through a portion of the variable volume pouch 400, and then again be inserted through a portion of the intervertebral cage 100, 300. In some embodiments, the passing of the deployment cable 806 through portions of the intervertebral cage 100, 300 and through portions of the variable volume pouch 400 can secure the variable volume pouch 400 to the intervertebral cage 100, 300.

In some embodiments, the variable volume pouch 400 can be connected to the intervertebral cage 100, 300 along the entire perimeter of the internal volume 126, and in some embodiments, the variable volume pouch 400 can be connected to the intervertebral cage 100, 300 at discrete points. In some embodiments, the variable volume pouch 400 can be connected to the intervertebral cage 100 at one point, two points, three points, four points, five points, six points, eight points, 10 points, 20 points, 50 points, or at any other or intermediate number of points. In some embodiments in which the variable volume pouch 400 is pre-inserted into the intervertebral cage 100, 300 and in which the opening 406 of the variable volume pouch 400 has been pre-positioned proximate to the proximal aperture 124 of the intervertebral cage 100, 300, the process 1300 can begin a block 1306. In some embodiments, the variable volume pouch 400 can be pre-affixed to the intervertebral cage 100, 300, and the process 1300 can begin at a block other than block 1306. In some embodiments in which the assembly of the intervertebral cage apparatus 500 is temporally separated from the use of the intervertebral cage apparatus 500, the process 1300 can terminate with block 1306.

In some embodiments of the process 1300 in which the assembly of the intervertebral cage apparatus 500 is temporally proximate to the use of the intervertebral cage apparatus 500, after the variable volume pouch 400 is affixed to the intervertebral cage 100, 300, the process 1300 can proceed to block 1308 wherein the intervertebral cage 100, 300 is expanded. In some embodiments, block 1308 can be preceded by processes for preparing the intervertebral space and for inserting the intervertebral cage apparatus 500. In some embodiments, these processes can include, for example, some or all of the steps of the process 1200 depicted in FIG. 12.

In some embodiments, the intervertebral cage 100, 300 can be expanded using any desired deployment technique and/or deployment device. In some specific embodiments, for example, the intervertebral cage can be expanded using a deployment system 800 comprising a deployment tool 802 and a deployment cable 806. In some embodiments, deployment of the intervertebral cage 100, 300 can result in a change in the dimensions of the intervertebral cage 100, 300 as measured along one or more of the longitudinal axis 104, the lateral axis 106, and/or the vertical axis 108.

After the intervertebral cage 100, 300 is expanded, the process 1300 proceeds to block 1310 wherein the variable volume pouch 400 is filled. In some embodiments, for example, the variable volume pouch 400 can be filled via the opening 406 at a variable volume pouch 400. In some embodiments, the variable volume pouch 400 can be filled via the opening 406 of the variable volume pouch and the proximal aperture 126 located in the proximal end 110 of the intervertebral cage 100, 300. In some embodiments, the variable volume pouch can be filled with, for example, a gaseous material, a liquid material, and/or a solid material. In some embodiments, the variable volume pouch 400 can be filled with a graph material which can comprise, for example, a solid material and specifically, a plurality of pieces of solid material. In some embodiments, these materials can comprise bone fragments and/or pieces of bones, and/or any biocompatible material.

In some embodiments, the variable volume pouch 400 can be filled with a desired amount of film material. In some embodiments, the desired amount of film material can be based on the desired size of the variable volume pouch 400 in its expanded state. Thus, in some embodiments, the desired size of the expanded state of the variable volume pouch 400 can determine the amount of film material. After the variable volume pouch 400 has been filled, steps can be taken to maintain the fill material within the variable volume pouch 400. In some embodiments, these steps can include, for example, sealing the opening 406, closing the opening 406, plugging the opening 406, or any other action that would prevent the film material from emptying out of the variable volume pouch 400.

FIG. 14 is a flowchart illustrating one embodiment of the process 1400 for preparing and/or using the intervertebral cage apparatus 900, 1100, which may or may not have a variable volume pouch 400. In some embodiments, the process 1400 can be performed before insertion of the intervertebral cage apparatus 900, 1100 into an intervertebral space, and in some embodiments, the process 1400 can be performed after insertion of the intervertebral cage apparatus into an intervertebral space.

The process 1400 begins at block 1402 wherein the deployment cable 806 is inserted through the proximal end 110 of the intervertebral cage 100, 300. In some embodiments, for example, the deployment cable 806 is inserted through the proximal end 110 of the intervertebral cage 100, 300 by inserting the deployment cable 806 through a first proximal opening 906, 1104, 1108. In some embodiments, the deployment cable 806 that is inserted through the first proximal opening 906, 1104, 1108 passes through the proximal end 110 of the intervertebral cage 100, 300 and into the internal volume 126 of the intervertebral cage 100, 300. In some embodiments, the deployment cable 806 that is inserted into the first proximal opening 906, 1104, 1108 passes into a channel and passes through all or portions of the intervertebral cage 100, 300. In some embodiments, the deployment cable 806 can be pre-inserted through the proximal end 110 of the intervertebral cage 100, 300, and the process 1400 can begin at a block other than block 1402. In some embodiments, the deployment cable 806 need not be inserted through the proximal end 110 of the intervertebral cage 100, 300, but is rather simply attached or affixed at or near the proximal end 110 of the intervertebral cage 100, 300.

After the deployment cable 806 is inserted through or affixed to the proximal end 110 of the intervertebral cage 100, 300, the process 1300 moves to block 1304 and the deployment cable 806 is inserted through the distal end 112 of the intervertebral cage 100, 300. In some embodiments, the deployment cable 806 can be inserted into the distal end 112 of the intervertebral cage 100, 300 by inserting the deployment cable 806 into and/or through a distal opening 902, 1110, 1112. In some embodiments, in which the deployment cable 806 passed through the proximal end 1110 of the intervertebral cage 100, 300 and into the internal volume 126, the deployment cable 806 can be inserted into the distal end 112 via the distal opening 902, 1110, 1112 from the internal volume 126. In some embodiments, in which the deployment cable 806 passes through a channel from the first proximal opening 906, 1104, 1108, the deployment cable 806 may be inserted through the distal end 1112 of the intervertebral cage 100, 300 by passing through a channel that travels through the distal end of the intervertebral cage. In some embodiments in which the deployment cable 806 has been pre-inserted through the proximal end 110 of the intervertebral cage 100, 300, the process 1400 can begin at block 1404. In some embodiments, the deployment cable 806 can be pre-inserted through the distal end 112 of the intervertebral cage 100, 300, and the process 1400 can begin at a block other than block 1404. In some embodiments in which the assembly of the intervertebral cage apparatus 900, 1100 is temporally separated from the use of the intervertebral cage apparatus 900, 1100, the process 1400 can terminate with block 1404.

In some embodiments, after the deployment cable 806 is inserted through the distal end 112 of the intervertebral cage 100, 300 the deployment cable 806 can be returned to the proximal end 110 of the intervertebral cage 100, 300. In some embodiments, the deployment cable 806 can return to the proximal end 110 of the intervertebral cage 100, 300 by inserting the deployment cable 806 into and/or through a second distal opening 902, 1110, 1112. After the deployment cable 806 has been inserted into and/or through the second distal opening 902, 1110, 1112, the deployment cable 806 passes through the distal end 112 of the intervertebral cage 100, 300 and into the internal volume 126 of the intervertebral cage 100, 300. In some embodiments, the deployment cable 806 that is inserted into the distal opening 902, 1110, 1112 passes into a channel and passes through all or portions of the intervertebral cage 100, 300.

After the deployment cable 806 returns to the proximal end 110 of the intervertebral cage 100, 300, the deployment cable 806 can be inserted through the proximal end 110 of the intervertebral cage 100, 300 by inserting the deployment cable 806 through a second proximal opening 908, 1102, 1106. In some embodiments, the deployment cable 806 that is inserted through the second proximal opening 908, 1102, 1106 passes from the internal volume 126 of the intervertebral cage 100, 300 and through the proximal end 110 of the intervertebral cage 100, 300. In some embodiments, the deployment cable 806 can be pre-inserted through the proximal end 110 of the intervertebral cage 100, 300. In some embodiments, the deployment cable 806 need not be inserted through the proximal end 110 of the intervertebral cage 100, 300, but can rather be simply attached or affixed at or near the proximal end 110 of the intervertebral cage 100, 300.

After the deployment cable 806 is inserted through or affixed to the proximal end 110 of the intervertebral cage 100, 300, the deployment cable 806 can be connected to the deployment tool 802, which can then be used to deploy the intervertebral cage 100, 300.

In some embodiments of the process 1400 in which the assembly of the intervertebral cage apparatus 900, 1100 is temporally proximate to the use of the intervertebral cage apparatus 900, 1100, the process 1400 proceeds to block 1406 wherein the intervertebral cage 100, 300 is expanded by applying a force to the intervertebral cage 100, 300 via the deployment cable 806. The force that is applied to the intervertebral cage 100, 300 via the deployment cable 806 can be generated using a variety of tools and/or techniques. In some embodiments, for example, in which the deployment cable 806 is part of an insertion system 800 including a deployment tool 802, the force can be applied to the intervertebral cage 100, 300 via the deployment cable 806 by using the control interface 804 to tension the deployment cable 806. In some embodiments, and as the force is applied to the intervertebral cage 100, 300 via the deployment cable 806, the user is provided feedback via the deployment tool 802 to allow the user to understand the status of the deployment. Specifically, in some embodiments, the deployment tool 802 can be configured to provide user feedback indicating that the further application of force to the intervertebral cage 100, 300 will result in the locking of the intervertebral cage 100, 300 in an expanded configuration. In some embodiments, for example, this feedback can comprise an audible, visual, and/or tactile signal that indicates that the intervertebral cage 100, 300 is nearing the locked and/or expanded configuration. In some embodiments, block 1406 can be preceded by processes for preparing the intervertebral space and for inserting the intervertebral cage apparatus 900, 1100. In some embodiments, these processes can include, for example, some or all of the steps of the process 1200 depicted in FIG. 12.

After the intervertebral cage 100, 300 is expanded by applying a force to the intervertebral cage 100, 300 via the deployment cable 806, the process 1400 proceeds to block 1408 wherein the intervertebral cage 100, 300 is locked in the expanded configuration. In some embodiments, in which the deployment cable 806 includes a locking feature 814, the intervertebral cage 100, 300 can be locked into the expanded configuration through the use of the locking feature 814 on the deployment cable 806. In one specific embodiment of how the locking feature 814 could be used in connection with the intervertebral cage 100, 300 to lock the intervertebral cage 100, 300 into an expanded configuration, the locking feature can comprise a member having a dimension and/or diameter larger than the diameter of the deployment cable 806. As the deployment cable 806 is retracted from the second proximal opening 908, 1104, 1108 to deploy the intervertebral cage 100, 300 the locking feature 814 can be moved through the proximal end 110 of the intervertebral cage 100, 300 and out the second proximal opening 908, 1104, 1108. In some embodiments, in which a locking feature 814 is used to secure the intervertebral cage 100, 300 in a expanded and/or locked configuration, the second proximal opening 908, 1104, 1108 can be configured to allow the locking feature 814 to pass through the proximal end 110 of the intervertebral cage 100, 300 and out the second proximal opening 908, 1104, 1108 but to prevent the locking feature 814 from retracting through the second proximal opening 908, 1104, 1108 and back into the proximal end 110 of the intervertebral cage 100, 300. Thus, in some embodiments, once the locking feature has been withdrawn from the proximal end 110 of the intervertebral cage 100, 300, via the second proximal opening 908, 1104, 1108, the locking feature can engage with portions of the second proximal opening 908, 1104, 1108 to secure the intervertebral cage 100, 300 in a locked and/or expanded configuration. In some embodiments, after the intervertebral cage 100, 300 has been locked in the expanded configuration, the force threshold can be exceeded, and the deployment cable 806 can break at the breakage point. In some embodiments, after the intervertebral cage 100, 300 is in the locked and/or expanded configuration, fill and/or graft material can be inserted into the internal volume 126 of the body 102 of the intervertebral cage 100, 300 via the proximal aperture 124.

A person of skill in the art will recognize that the process 1300 and 1400 can include more or fewer steps than those outlined above. A person of skill in the art will further recognize that the above outlined steps of processes 1300 and 1400 can be performed in any desired order, and can include substeps or subprocesses. A person of skill in the art will further recognize that the specific methods of locking the intervertebral cage 100, 300 into an expanded configuration are not limited to the specific embodiments enumerated herein, but that a wide variety of techniques and devices can be used to lock the intervertebral cage 100, 300 in an expanded and/or locked configuration. A person of skill in the art will further recognize that the processes depicted in FIGS. 12, 13, and 14 can be combined, and that thus an intervertebral cage 100, 300 can be used with both the variable volume pouch 400 and the deployment cable 806.

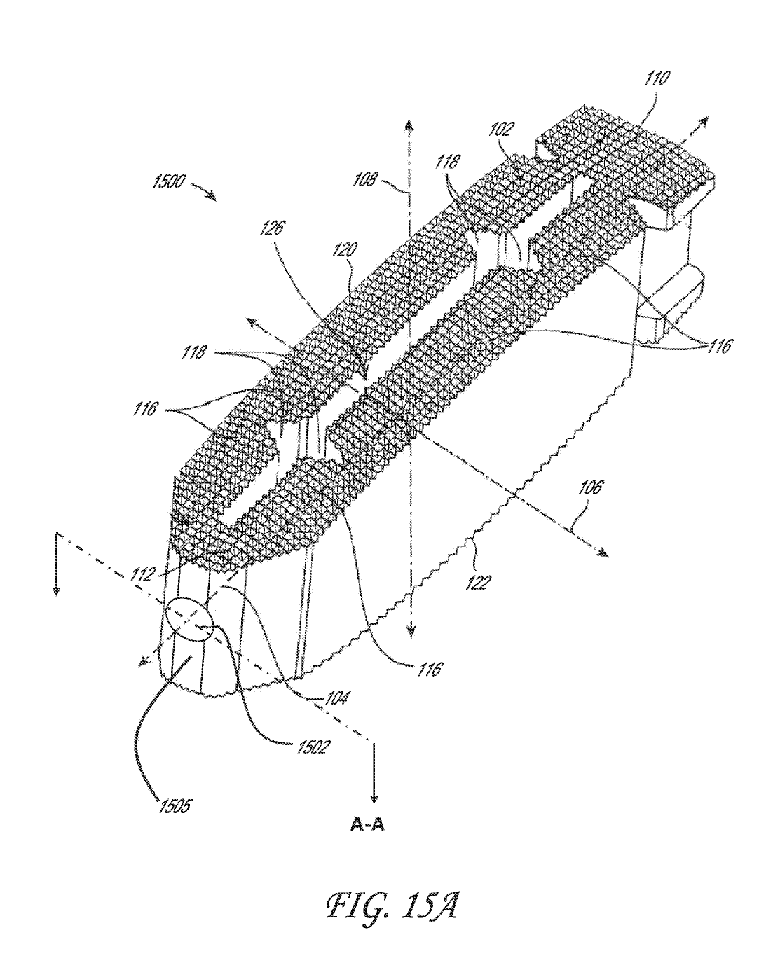

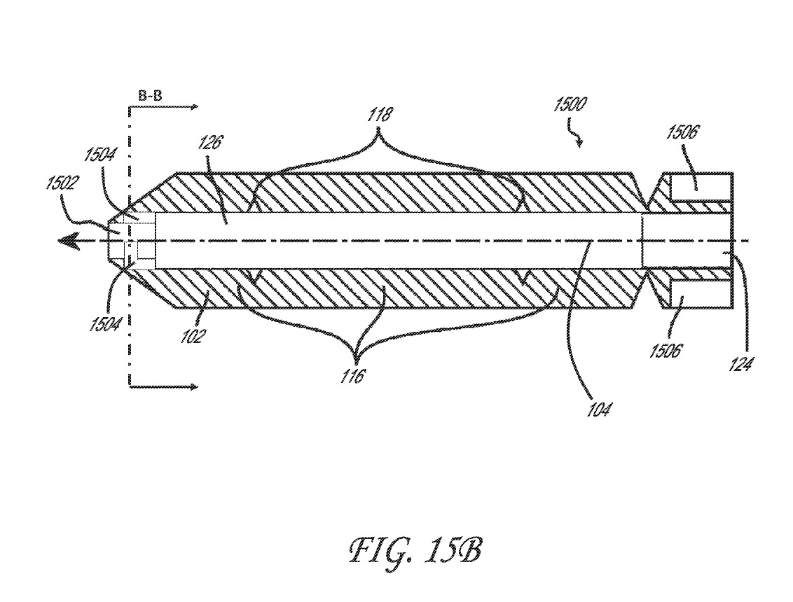

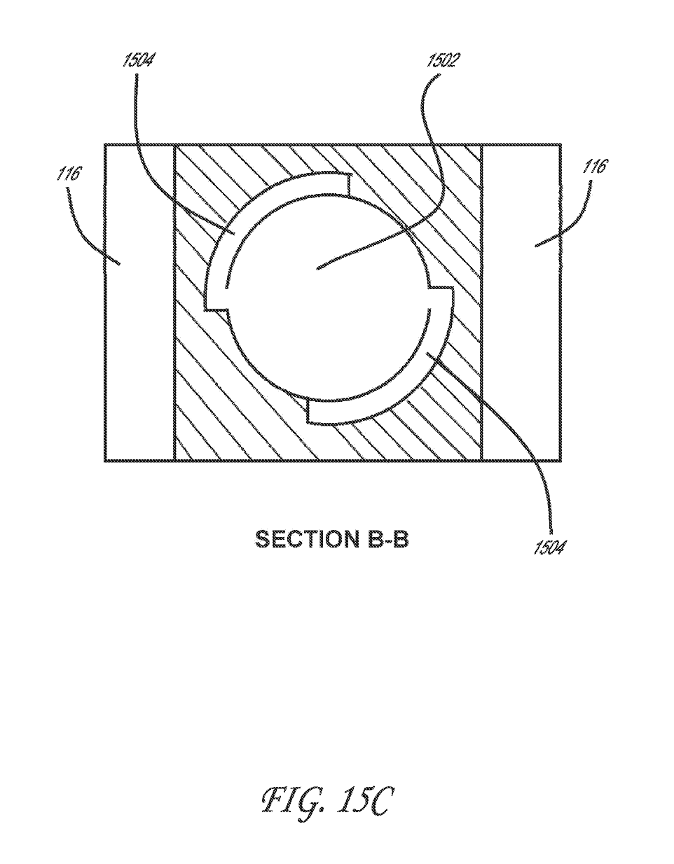

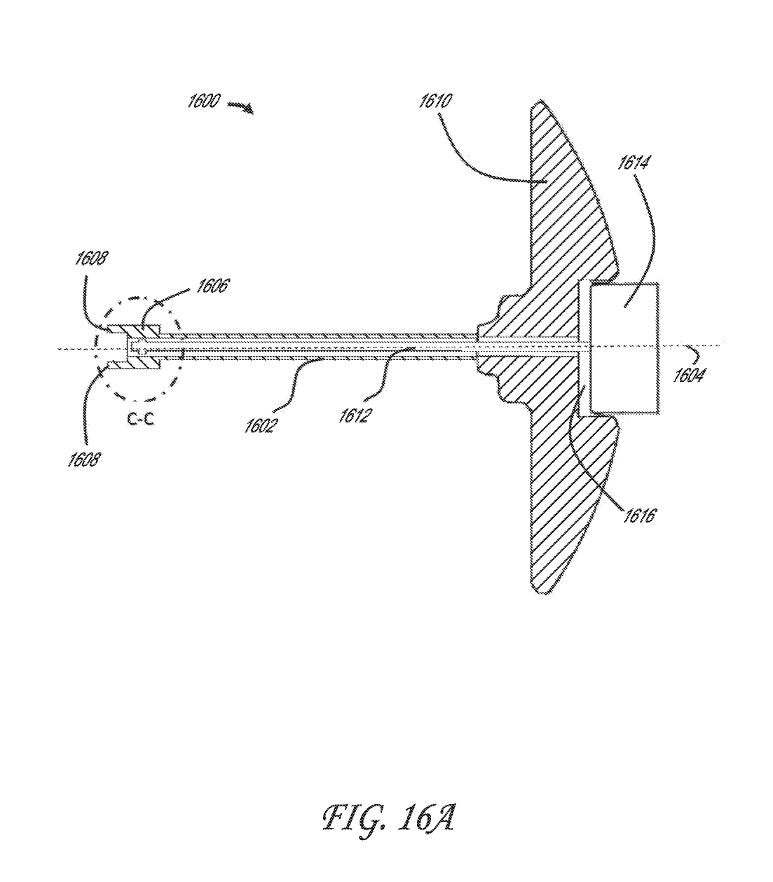

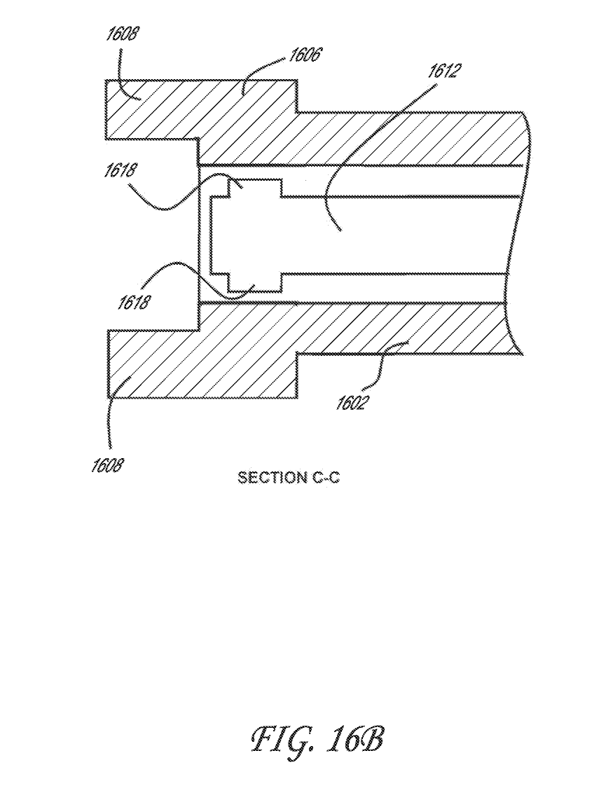

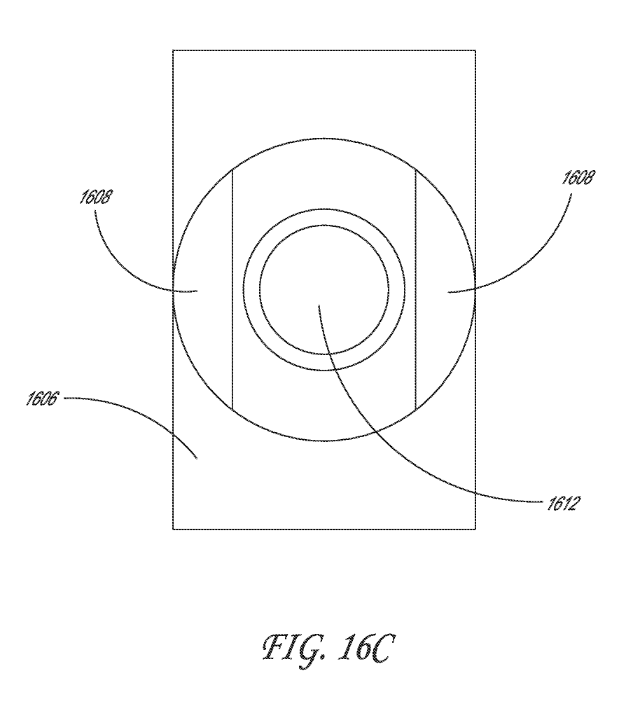

FIGS. 15A-15C are illustrations of an embodiment of an intervertebral cage apparatus 1500 which has a distal aperture 1502 located at a distal end of the body 102. With reference to FIG. 15A which is a perspective view of the intervertebral cage apparatus 1500, the distal aperture 1502 is centered on the longitudinal axis 104 although in alternative embodiments the aperture 1502 may be offset from the axis 104. In the illustrated embodiment, the distal aperture 1502 has a diameter less than that of the proximal aperture 124 and incorporates a coupling mechanism along its inner surface. In certain embodiments, the coupling mechanism takes the form of a bayonet mount. As shown in FIG. 15B which is a cross-sectional view of the intervertebral cage apparatus 1500 along Section A-A (shown in FIG. 15A), the distal aperture may have two or more slots 1504 configured to receive two or more pins 1618 on a distal end of an implantation tool 1600 (described further with respect to FIGS. 16A-16C).

In the illustrated embodiment, the slots 1504 are “L-shaped” slots formed along the inner surface of the distal aperture such that a first portion of the slot extends from a proximal end of the distal aperture 1502 distally to a location between the proximal end and distal end of the aperture 1502. As shown more clearly in FIG. 15C which is a cross-sectional view of the intervertebral cage apparatus 1500 along Section B-B (shown in FIG. 15B), a second portion of the slot 1504 then extends radially along the inner circumference of the inner surface of the distal aperture. The radial extension can be about 45 degrees to about 135 degrees about the longitudinal axis 104. In the illustrated embodiment, the circumferential extension is about 90 degrees. In some embodiments, fewer or greater slots 1504 may be used. Additionally, in some embodiments, the slots 1504 may be placed such that the first portion of the slot 1504 is centered on a plane formed by axes 104 and 108. This could advantageously allow larger pins 1618 to be used thereby reducing localized stresses and strains when deploying the device.

In other embodiments, slots 1504 of the distal aperture 1502 have no second portion such that the first portion runs entirely from a proximal end of the aperture 1502 to a distal end of the aperture 1502 allowing for the pins 1618 to wholly pass therethrough. In such embodiments, the pins 1618 of the implantation tool can instead be used to engage and abut a distal face 1505 of the intervertebral cage apparatus 1500. In yet other embodiments, the distal aperture 1502 has a diameter which is equal to, or greater than, the diameter of the proximal aperture 124. Furthermore, it is contemplated that in other embodiments, other types of coupling mechanisms may be used to couple the implantation device with the body 102, such as, but not limited to, a press fit, an interference fit, a friction fit, threads, and other coupling mechanisms known in the art.

With reference to FIG. 15B, the proximal end 110 of the body 102 has cutouts 1506 configured to receive mating portions 1608 of an implantation tool 1600 shown in FIGS. 16A-16C. In the illustrated embodiment, two cutouts 1506 are located along the outer perimeter of the proximal end 110. In other embodiments, a different number of cutouts 1506 can be used and is not limited to placement along the outer perimeter of the proximal end 110 of the body 102.

FIGS. 16A-16C are illustrations of an embodiment of an implantation tool 1600 which can be used to convert the intervertebral cage apparatus 1500 or other cage apparatuses described herein from an unexpanded position to a expanded position. With reference to FIG. 16A which is a partial cross-section of an embodiment of an implantation tool 1600, the implantation tool 1600 has an outer cannula 1602 extending between a proximal end and a distal end of the tool 1600 and centered on luminary axis 1604. At the distal end of outer cannula 1602 is a connector 1606 configured to contact the proximal end 110 of body 102. As shown more clearly in FIG. 16C, which is a view of the distal end of the implantation tool 1600, in one embodiment the connector 1606 has dimensions which are equal to, or greater than, the dimensions of the proximal end 110 of body 102 such that the connector 1606 advantageously distributes any contact pressure over the entire surface area of the proximal end 110. In some embodiments, connector 1606 has two mating portions 1608 such as teeth protruding distally from the connector 1606 which are configured to be inserted into and engage cutouts 1506 on the proximal end 110 of the body 102. In other embodiments, connector 1606 may have fewer or greater mating portions 1608 depending on the amount of cutouts 1506 on the proximal end 110 of the body 102. Once engaged, the mating portions 1608 are configured to directly link the rotation of the body 102 with the rotation of the outer cannula 1602 thereby providing a user of the implantation tool 1600 direct control of the rotation of the body 102 during an implantation procedure.

At the proximal end of the outer cannula 1602 is a handle 1610 configured to be held by a user of the implantation tool 1600. The handle 1610 is directly attached to the outer cannula 1602 such that rotation of the handle 1610 also causes rotation of the outer cannula 1602. As such, a user of the implantation tool 1600 can advantageously control the rotation of the body 102 through the handle 1610. Implantation tool 1600 also has an internal shaft 1612 centered about the luminal axis 1604 which is both slidably translatable and slidably rotatable within the outer cannula 1602. In the illustrated embodiment, the internal shaft 1612 is directly attached to control member 1614 such that rotation and translation of control member 1614 rotates and translates the internal shaft 1612. In this embodiment, the control member 1614 is wholly received within an aperture 1616 in the handle. In other embodiments, the aperture is sized only to receive the internal shaft 1612 such that the control member 1614 remains outside of the handle. Control member 1614 may have raised ridges, protrusions, texturing, grips, or other mechanisms to assist a user of the device to rotate and translate the control member 1614.

In some embodiments, at the distal end of shaft are pins 1618 which correspond to the coupling mechanism in the form of slots 1504 located on the distal aperture 1502 of the intervertebral cage apparatus 1500. Since shaft 1612 is slidably translatable and slidably rotatable within the outer cannula 1602, the shaft 1612 can be both be translated and rotated to engage the “L-shaped” slot 1504 of the distal aperture 1502 while a counter-force is applied to the body 102 via the outer cannula 1602 due to the engagement of the mating portions 1608 with the cutouts 1506.

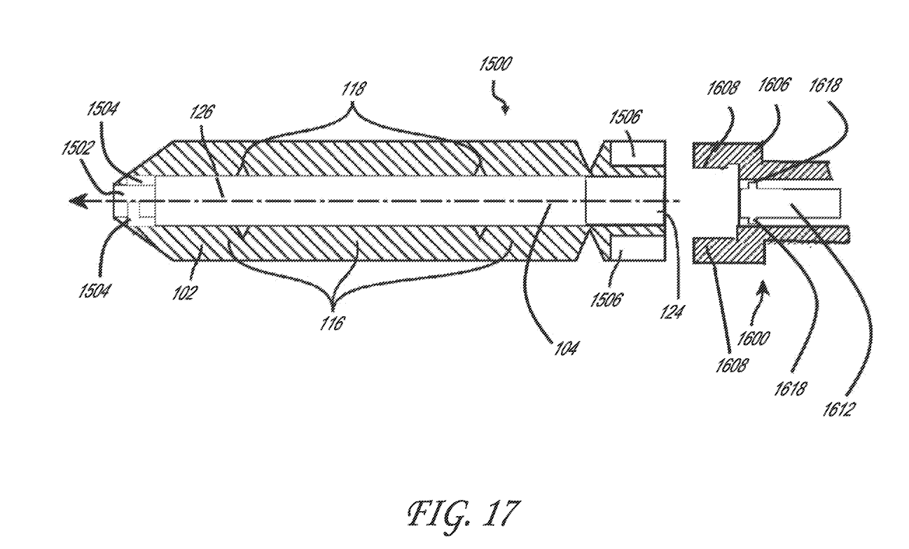

FIG. 17 illustrates one method by which the implantation tool 1600 can be used to convert the intervertebral cage apparatus 1500 and any other such apparatus described herein from an unexpanded position to an expanded position. In the illustrated embodiment, a shaft 1612 with pins 1618 and an intervertebral cage apparatus 1500 with a distal aperture 1502 containing slots 1504 is used. During a first step, the implantation tool 1600 is advanced towards the proximal end 110 of the intervertebral cage apparatus 1500 in the unexpanded configuration such that the connector 1606 is placed adjacent to and in contact with the proximal end 110 of the body 102. During this advancement process, mating portions 1608 are simultaneously inserted into and engage the cutouts 1506 thereby linking the rotation of the body 102 with the rotation of the outer cannula 1602.

During a second step, the shaft 1612 is then slidingly advanced distally through the outer cannula 1602 and into the intervertebral cage apparatus 1500. The shaft advances first through the proximal aperture 124, then through the internal volume 126, and finally placed adjacent to and in contact with the trailing edge of the distal aperture 1502. In this embodiment, since the distal aperture 1502 has slots 1504 which correspond to the pins 1618 at the distal end of the shaft 1612, the shaft 1612 can be further advanced into the distal aperture 1502 by following the profile of the slot 1504. The shaft 1612 can then be rotated such that the shaft 1612 is engaged with the distal aperture 1502. In this engaged position, the shaft 1612 and body 102 are linked such that translation of the shaft 1612 results in translation of the body 1502. Note that the labeling of the above steps as “first” and “second” is used solely to describe one method of deploying the intervertebral cage apparatus 1500 and other cage apparatuses described herein. In other embodiments, this sequence can be reversed such that the second step is completed before the first step.

In embodiments of the intervertebral cage apparatus 1500 having slots 1504 which extend throughout the length of the distal aperture 1502, the shaft is advanced wholly through the distal aperture 1502. Upon the pins 1618 being distal the distal face 1505 of the body 102, the shaft 1612 is rotated and retracted such that the pins 1618 are abutting a distal face 1505.

Additionally, the above described steps can either be performed prior to or after insertion of the intervertebral cage apparatus 1500 into the intervertebral space. In embodiments where the above-described steps are performed prior to insertion into the intervertebral space, the implantation tool 1600 is used to deliver the device into the space. In embodiments where the above-described steps are performed after insertion into the intervertebral space, a separate tool may be used to deliver the device into the space.

During the third step, after the shaft 1612 has been engaged with the distal aperture 1502, a force is applied, in the distal direction, to the proximal end 110 of the body 102 while the shaft 1612 is held in place. The force applied to the proximal end 110 causes the body 102 to convert from the unexpanded position to the expanded position due to deformation along living hinges 118. In an alternative embodiment, a force is applied, in the proximal direction, to the distal end of the body 102 at the distal aperture 1502 while the outer cannula 1602 is held in place to convert the body 102 from an unexpanded position to an expanded position.

During the final step, the shaft 1612 is rotated to disengage pins 1618 from the “L-shaped” slot of the distal aperture 1502. The shaft 1612 is then slidingly retracted from the intervertebral cage apparatus 1500 such that the shaft 1612 is removed from the body 102. The connector 1606 may then be retracted such that the mating portions 1608 are removed from cutouts 1506. The tool may then be removed from the intervertebral space and the body of the patient.

Vertically Expandable Intervertebral Cage

FIG. 18 is an illustration of an embodiment of the intervertebral cage 1800 in an expanded configuration. The intervertebral cage 1800 has both a front panel 1802 and a circuitous body 1805. In an expanded configuration, front panel 1802 is configured to provide structural support, in the form of a strut, for the cage 1800. In one embodiment, the front panel 1802 includes a living hinge 1810 positioned equidistant from the top edge 1812 and the bottom edge 1814 which subdivides the front panel 1802 into both a top section 1816a and a bottom section 1816b. In other embodiments, the living hinge 1810 may be placed closer to the top edge 1812 or to the bottom edge 1814 depending upon the geometry desired in the unexpanded and expanded configurations. Furthermore, in yet other embodiments, more than one living hinge can be included on the front panel 1802.

In some embodiments, the top section 1816a and the bottom section 1816b are separate units which are rotatably attached at hinge 110 to form front panel 102. In those embodiments, rotatable attachment of the top and bottom sections 1816a, 1816b can be accomplished through materials allowing for elastic deformation such as a hinge formed via a reduced thickness or living hinge of the front panel 1802 along the living hinge 1810 which is configured to allow deformation along living hinge 1810.

In one embodiment, such as that illustrated in FIG. 18, the front panel 1802 has an aperture 1818 substantially centered on the front panel 1802. In other embodiments, the front panel 1802 has multiple apertures located on both the top section 1816a and bottom section 1816b. Aperture 1818 can be configured to allow a distal part of an implantation device to enter through the trailing end 1808 of the intervertebral cage 1800 and pass through aperture 1818 such that the distal part of the implantation device is distal the front panel 1802. In embodiments with multiple apertures on the top section 1816a and the bottom section 1816b, the apertures can be configured to allow a guide wire to be inserted through the trailing end 1808 of the intervertebral cage 1800 through a first aperture and returned to the trailing end 1808 through a second aperture. The implantation device or guide wire can be used to apply the force to convert the intervertebral cage 1800 from an unexpanded configuration to an expanded configuration.

As another example, an inflatable device, such as an inflatable bladder, can be placed within the interior volume. The inflatable device can be inflated such that the inflatable device increases in volume within the interior volume. The inflatable device can contact portions of the intervertebral cage 1800 such that a force is applied on the intervertebral cage to deploy the cage 1800. Such methods and devices are described in more detail in U.S. patent application Ser. No. 14/422,750, the entire contents of which is hereby incorporated by reference.

In the illustrated embodiment, the top and bottom sections 1816a, 1816b are generally of rectangular shape notwithstanding the aperture 1818. In such a configuration, the top edge 1812 and the bottom edge 1814 generally remain parallel. In other embodiments, the top and bottom sections are not rectangular shaped but rather wedge shaped such that the top and bottom edges 1812, 1814 are not parallel. These embodiments can be used when the two surfaces requiring support are oblique and different heights are necessary. Other shapes may include quadrilaterals such as, but not limited to, squares, rectangles, parallelograms, and trapezoids. Shapes may also include polygons with more than four sides, partial ellipses such as semi-circles, and any other shape as may be chosen by one of skill in the art.

It should be apparent to one of skill in the art that the panels of the intervertebral cage 1800 could form integral units with adjacent panels through the use of a living hinge or could be separate from adjacent units and rotatably attached via attachment mechanisms described above. In some embodiments of the device, both living hinges and other attachment mechanisms are simultaneously used. This could allow the device to be assembled post-manufacturing and potentially provide cost savings. In other embodiments, living hinges are used throughout the entire device.

Operation

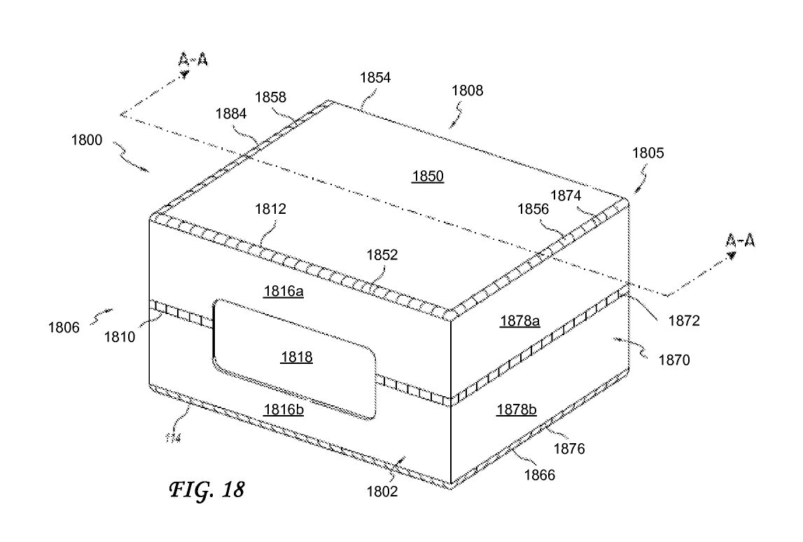

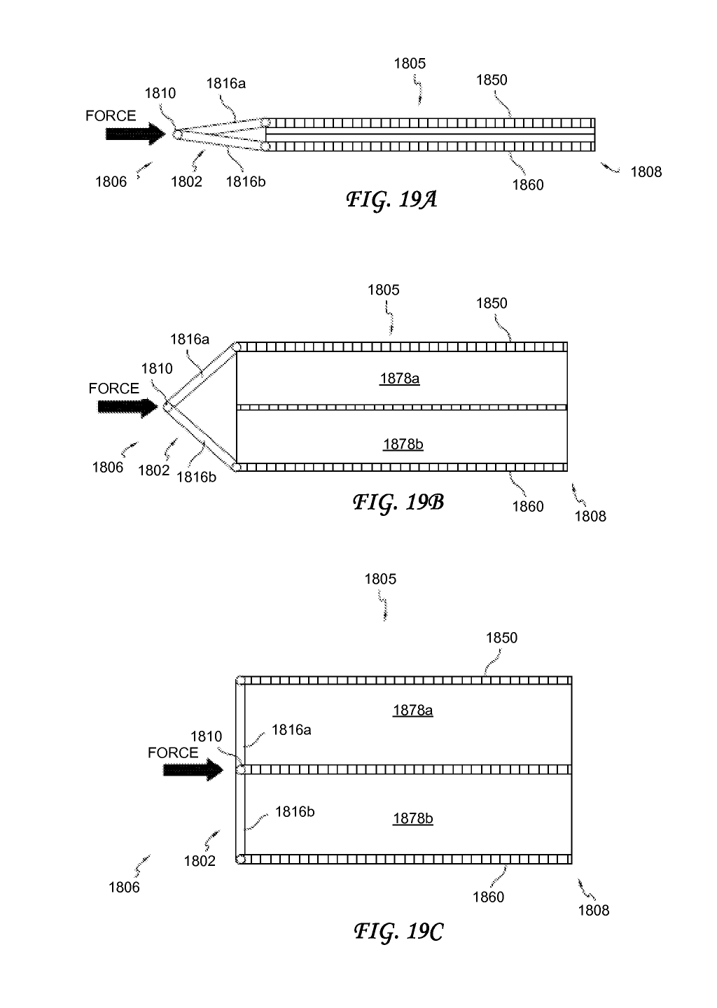

As discussed above, the panels and sections of the intervertebral cage 1800 are rotatably attached via the living hinges to adjacent panels and sections. As such, the separate panels of the intervertebral cage 1800 can rotate from an unexpanded configuration to an expanded configuration. FIGS. 19A-19C is a view from the left side of the intervertebral cage 1800 which illustrates one non-limiting method of converting the intervertebral cage 1800 from an unexpanded configuration to an expanded configuration. Three separate configurations are shown: the unexpanded configuration (shown in FIG. 19A), an intermediate configuration (shown in FIG. 19B), and the expanded configuration (shown in FIG. 19C).

With reference to FIG. 19A, while in the unexpanded configuration, the front panel 1802 is collapsed such that top section 1816a and bottom section 1816b form the sides of a wedge with living hinge 1810 forming the tip of the wedge. In other embodiments, the side panels are collapsed inwardly. In the embodiment shown, the living hinge 1810 extends outwardly in a distal direction thereby providing the intervertebral cage 1800 with a wedge-shaped or tapered leading end 1806. During an implantation procedure, since the front panel 1802 is the initial portion of the intervertebral cage 1800 that enters the surgical site and the vertebral space, this wedge-shaped or tapered configuration facilitates insertion of the intervertebral cage 1800 into the patient during an implantation procedure. First, because of the wedge shape, the user is assisted in centering the cage 1800 within the space formed by the two vertebral end plates as the user advances the cage 1800 into this space. Second, since the intervertebral cage 1800 is at a reduced height in the unexpanded configuration, there is a reduced likelihood that portions of the cage 1800, such as the top and/or bottom panels 1850, 1860, will contact the end plates thereby hindering advancement of the cage 1800 during the procedure.

In some embodiments, conversion the device from the unexpanded configuration to the expanded configuration can be performed by applying a force in the direction of the trailing edge 1808, to the front panel 1802. This can be accomplished by pulling the front panel 1802 while inhibiting any translation of the device in a plane parallel to the vertebral end plates (i.e., by applying, for example, a counter-force on the top and bottom plates 1850, 1860). Due to both the force on the front panel 1802 and the rotatable attachment of the top and bottom sections 1816a, 1816b, the living hinge 1810 is pulled towards the trailing end 1808. This motion increases the angle formed between the top section 1816a and the bottom section 1816b thereby causing a vertical expansion of the cage 1800.

Note that the cage 1800 can also be opened using other methods. For example, a force can instead be placed on the circuitous body 1805 towards the leading end 1806 while inhibiting the front panel 1802, or more specifically the living hinge 1810, from translating in the same direction. In another example the forces may instead be applied to the side panels in a direction opposite that in which they are collapsed. Thus, if collapsed inwardly towards the interior volume 1890, the separate forces can be applied on each side panel in a direction away from the interior volume 1890. If collapsed outwardly away from the interior volume 1890, the separate forces can be applied on each side panel in a direction towards the interior volume 1890. In another example an upwards vertical force may be applied to the bottom surface of the top panel and a downwards vertical force may be applied to the top surface of the bottom panel to commence the conversion process. Some or all of the methods described above can be combined together during the process of converting the cage 100 from an unexpanded configuration to an expanded configuration.

### VARIATIONS, MODIFICATIONS, AND COMBINATIONS

Various modifications to the implementations described in this disclosure may be readily apparent to those skilled in the art, and the generic principles defined herein may be applied to other implementations without departing from the spirit or scope of this disclosure. Moreover, any of the steps described herein can be performed simultaneously or in an order different from the steps as ordered herein. Moreover, as should be apparent, the features and attributes of the specific embodiments disclosed herein may be combined in different ways to form additional embodiments, all of which fall within the scope of the present disclosure. Thus, the disclosure is not intended to be limited to the implementations shown herein, but is to be accorded the widest scope consistent with the principles and features disclosed herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

What is claimed is:

1. A method for making an expandable intervertebral cage with living hinges using 3D printable materials for placement between adjacent vertebrae, the method comprising:
providing 3D data of the expandable intervertebral cage to a 3D printer, the expandable intervertebral cage includes a circuitous body having a plurality of side segments rotatably attached by integral living hinges configured to flex or deform during the transition of the circuitous body from an unexpanded configuration to an expanded configuration; and
printing the plurality of side segments and integral living hinges of the circuitous body using one or more 3D printable materials.

2. The method of claim 1, wherein the one or more 3D materials is selected from the group consisting of: thermoplastics, photopolymers, metal powders, eutectic metals, titanium alloys and combinations thereof.

3. The method of claim 1, wherein the one or more 3D material is selected from the group consisting of: a natural biocompatible material, a synthetic biocompatible material, a metallic biocompatible material, adaptive material, 4D printing, and combinations thereof.

4. The method of claim 1, wherein the one or more 3D material is selected from the group consisting of: polyetherketone (PEK), polyetherimide (PEI), such as Ultem, ultrahigh molecular weight polyethylene (UHMPE), polyphenylene, polyether-ether-ketone (PEEK), comprise a memory PEEK material such as, for example, PEEK Altera, and combinations thereof.

5. The method of claim 1, wherein the intervertebral cage includes an external coating selected from the group consisting of plasma spray, hydroxyapatite coating, biologics, antibiotics, drug or gene therapy, nanotechnology platform(s), and combinations thereof.

6. The method of claim 1, wherein the integral living hinges comprise a relatively flexible 3D printable material and the side segments comprise a relatively rigid 3D printable material.

7. The method of claim 1, wherein the relatively flexible 3D printable material comprises a non-metallic material and the relatively rigid 3D printable material comprises a metallic material.

8. The method of claim 1, wherein the 3D data is predefined data of standard cage sizes.

9. The method of claim 1, wherein the 3D data is 3D imaging data, the method further comprising pre-operatively imaging adjacent vertebrae of a patient to generate 3D imaging data.

10. The method of claim 5, wherein the external coating is a 3D printable material.

11. The method of claim 1, wherein the living hinges comprise narrowed portions of the circuitous body and/or cutouts into the circuitous body configured to allow localized flexure or deformations of the circuitous body.

12. The method of claim 1, wherein the circuitous body includes proximal and distal ends oppositely disposed along a lateral axis and in the unexpanded configuration the proximal and distal ends are at a maximum separation and in the expanded configuration the proximal and distal ends are closer together, the expandable intervertebral cage configured to horizontally expand from the unexpanded configuration to the expanded configuration between adjacent vertebrae.

13. The method of claim 1, further comprising printing a top panel and a bottom panel, wherein each top and bottom panel rotatably attached by integral living hinges to one or more side segments, the expandable intervertebral cage configured to vertically expand from the unexpanded configuration to the expanded configuration between adjacent vertebrae.

14. The method of claim 1, wherein the expandable intervertebral cage further comprises a variable volume pouch positionable within an interior volume of the intervertebral cage.

15. The method of claim 1, wherein the expandable intervertebral cage further comprises a deployment cable coupled to the circuitous body and configured to apply a force to the circuitous body to transition the circuitous body from the unexpanded configuration to the expanded configuration.

16. The method of claim 1, wherein the expandable intervertebral cage further comprises a deployment tool coupled to the circuitous body and configured to apply a force to the circuitous body to transition the circuitous body from the unexpanded configuration to the expanded configuration.

17. The method of claim 1, wherein the expandable intervertebral cage is configured for positioning between end plates of two vertebrae and further configured to transition from an unexpanded configuration to an expanded configuration resulting in a change of the dimensions and shape of the expandable intervertebral cage and increasing a modifiable interior volume of the expandable intervertebral cage.

18. A method for making and using a patient specific expandable intervertebral cage with living hinges using 3D printable materials for placement between adjacent vertebrae, the method comprising:
pre-operatively imaging adjacent vertebrae of the patient to generate 3D imaging data;
providing the 3D data to a 3D printer;
printing an expandable intervertebral cage using one or more 3D printable materials, the expandable intervertebral cage includes a circuitous body having a plurality of side segments rotatably attached by integral living hinges configured to flex or deform during the transition of the circuitous body from an unexpanded configuration to an expanded configuration; and
surgically positioning the expandable intervertebral cage between the adjacent vertebrae; and
expanding the expandable intervertebral cage from the unexpanded configuration to the expanded configuration between the adjacent vertebrae.

19. The method of claim 1, wherein the one or more 3D material is selected from the group consisting of: thermoplastics, photopolymers, metal powders, eutectic metals, titanium alloys, a natural biocompatible material, a synthetic biocompatible material, a metallic biocompatible material, polyetherketone (PEK), polyetherimide (PEI), such as Ultem, ultrahigh molecular weight polyethylene (UHMPE), polyphenylene, polyether-ether-ketone (PEEK), comprise a memory PEEK material such as, for example, PEEK Altera, and combinations thereof.

20. A system for deploying an expandable intervertebral cage with living hinges using 3D printable materials for placement between adjacent vertebrae, the system comprising:
an expandable intervertebral cage made of one or more 3D printable materials configured to transition from an unexpanded configuration to an expanded configuration, having a proximal end and a distal end, the expandable intervertebral cage includes a circuitous body having a plurality of side segments rotatably attached by integral living hinges configured to flex or deform during the transition of the circuitous body from the unexpanded configuration to the expanded configuration; and
a variable volume pouch positionable within the intervertebral cage, the variable volume pouch being configured to move the expandable intervertebral cage from the unexpanded configuration to the expanded configuration.
